(19) United States
(12) Patent Application Publication
(10) Pub. No.: US 2025/0059492 A1
(43) Pub. Date: Feb. 20, 2025

(54) SYSTEMS, DEVICES, AND METHODS FOR CONDITIONAL EXECUTION OF AUTOMATIC CELL PROCESSING

(71) Applicant: Cellares Corporation, South San Francisco, CA (US)

(72) Inventors: Marc A. BEBAN, Redwood City, CA (US); Fabian G. GERLINGHAUS, San Francisco, CA (US); Victoria Patricia Hodgson ZIEBARTH, San Bruno, CA (US); Daniele MALLEO, Menlo Park, CA (US); Amanda M. MARCHIANDO, San Francisco, CA (US); Yangzi I. TIAN, Sunnyvale, CA (US)

(21) Appl. No.: 18/807,699

(22) Filed: Aug. 16, 2024

Related U.S. Application Data
(60) Provisional application No. 63/520,312, filed on Aug. 17, 2023.
(60) Provisional application No. 63/520,313, filed on Aug. 17, 2023.

(51) Int. Cl. C12M 1/36; C12M 1/34

(52) U.S. Cl. CPC C12M 41/48; C12M 41/46

(57) ABSTRACT

The present disclosure relates to systems, devices, and methods for conditional execution within a cell processing system. In an embodiment, the present disclosure relates to a method of automatically executing a pre-programmed workflow defining a set of cell processing steps, determining whether a condition has been met based upon at least one measured parameter, and continuing, modifying, or stopping the workflow based upon the determination.

### CROSS REFERENCE TO RELATED APPLICATIONS

This application claims priority to U.S. Provisional Patent Application Ser. No. 63/520,313 filed Aug. 17, 2023, and U.S. Provisional Patent Application Ser. No. 63/520,312 filed Aug. 17, 2023, the contents of which are incorporated herein by reference in their entirety for all purposes.

### TECHNICAL FIELD

The present disclosure relates to systems, devices, and methods for cell processing, for example, conditional execution of automatic cell processing.

### BACKGROUND

Cell therapies involve collecting cells from an individual, processing the cells, and utilizing the processed cells to achieve a clinical response in the same or a different individual. Cell processing, which can include growing or culturing cells, is a complex workflow that involves multiple steps, each of which can take multiple days to complete and often requires intermittent measurement steps to evaluate the status of a given cell processing step. Many current manufacturing processes are performed manually, which is operationally inefficient and labor intensive. Even platforms described as automated cell processing in a closed system generally rely on pre-configured instrumentation that limit operational flexibility and do not reliably perform successful cell processing without operator support throughout. The lack of operational flexibility often precludes the ability to modify or continue the workflow based on the measurements obtained during the measurement steps. For example, some cell processing workflows intended to grow therapeutic cells may be severely delayed, or aborted entirely, when a measurement does not match a quality control metric and the closed system has inadequate flexibility to rectify the issue without human intervention. The issues compound when multiple cell processing workflows are run in parallel with staggered timing. Accordingly, additional systems and methods for automatically executing cell processing workflows are desirable.

### SUMMARY

The present disclosure relates generally to systems, devices, and methods for conditional execution of cell processes within an automated cell processing system. In general, a method for cell processing may include automatically executing a pre-programmed workflow defining a set of cell processing steps. The execution of at least one of the cell processing steps may include measuring at least one parameter based upon a set of preprogrammed conditions and corresponding outcomes for that step. The method may further include determining whether a condition has been met based upon the at least one measured parameter and the workflow may be continued, modified, or stopped based upon the determination. The set of cell processing steps may include one or more of enrichment, selection, activation, expansion, perfusion, sampling, and harvesting. In some variations two or more cell processing steps may be executed simultaneously. The parameter may include one or more of a total cell count value, a target cell count value, a cell concentration value, a cell recovery value, a cell diameter value, a cell viability value, a glucose value, a lactate value, a dissolved oxygen value, a pH value, a chimeric antigen receptor expression value, and a transgenic T cell receptor expression value. The parameter may be measured at a predetermined time interval.

The condition may define a threshold value for the parameter. A cell processing step may be executed upon a determination that the measure parameter meets the condition. In some variations cells may be transferred from a first module to a second module upon a determination that the measured parameter meets the condition. Upon a determination that the measured parameter does not meet the condition, a notification to a user may be generated, the workflow may be stopped, and/or at least one cell processing step may be repeated.

In some variations, a method for automated cell processing may include receiving a workflow, automatically executing the workflow, automatically measuring at least one parameter within a cartridge within a work cell, comparing the at least one measured parameter to a predefined condition, and automatically continuing, modifying, or stopping the workflow based upon the comparison. In some variations, an alert may be generated, the workflow may be stopped, and/or at least one cell processing step may be repeated upon the comparison indicating that the measure parameter does not meet the predefined condition. Continuing the workflow may include ending a first cell processing step and executing a second cell processing step. The cell processing steps may be selected from the group consisting of washing, isolating, purifying, enriching, diluting, and growing cells. The at least one parameter may be selected from the group consisting of cell count, cell diameter, cell concentration, cell viability, glucose, lactate, cell recovery, dissolved oxygen, and pH.

The workflow may be automatically executed by a robot. The workflow and or the predefined condition may each be pre-programmed into a controller. Measuring at least one parameter may be performed by an analytical instrument. In some variations a sample of the cells within the cartridge may be transferred to an analytical instrument.

In some variations, a method for automated cell processing may include performing a first cell processing step on cells within a cartridge, measuring a first parameter based on the first cell processing step, comparing the measured first parameter to a first set of preprogrammed conditions to determine whether a first condition has been met, after determination that the first condition has been met performing a second cell processing step on the cells within the cartridge, measuring a second parameter based on the second cell processing step, and comparing the measured second parameter to a second set of preprogrammed conditions to determine whether a second condition has been met. The first cell processing step and second cell processing step may include enrichment, selection, activation, or expansion. In some variations, each of the first and second cell processing further include cell perfusion, cell sampling, or cell harvesting. Each of the first and second cell processing steps may be executed automatically by a robot. In some variations, the first cell processing step may be performed by a first instrument and the second cell processing step may be performed by a second instrument, wherein the first and second instruments may be collocated within a workcell. The first and second cell processing steps may each be performed at pre-defined time intervals.

In some variations, each of the first and second conditions may include a total cell count value, a target cell count value, a cell concentration value, a cell recovery value, a cell diameter value, a cell viability value, a glucose value, a lactate value, a dissolved oxygen value, a pH value, a chimeric antigen receptor expression value, or a transgenic T cell receptor expression value. A user may pre-program each of the first and second conditions using a controller. A user alert may be generated if either of the first or second condition is not met.

The systems described herein may include a controller for conditionally executing an automated cell processing method that includes at least one pre-programmed workflow defining a set of cell processing steps and a set of pre-programmed conditions and corresponding outcomes for use in modifying at least one of the cell processing steps. At least one of the pre-programmed conditions may be selectable by a user from the pre-programmed set. The controller may be configured to receive input from a user to pre-program one or more workflow; condition, and outcome. The controller may be further configured to generate an alert to a user if at least one of the pre-programmed conditions is not met. The controller may be configured to control a robot where the robot is configured to automatically execute the pre-programmed workflow. In some variations, the controller may be in communication with a sterile liquid transfer instrument. The system can further include a cell sampling system configured to perform one or more measurements of a cell solution and/or one or more instruments configured to perform one or more cell processing steps.

Also described herein are methods directed to automatically growing cells. A method for automated cell growth may comprise delivering a cartridge to a first instrument bay of a cell processing workcell. The cartridge may have cells therein and may comprise at least a first module and a second module. The workcell may comprise a robot, a sterile liquid transfer instrument, and a controller comprising at least one pre-programmed workflow defining at least a first cell processing step and a second cell processing step. The method may further comprise performing at least one of the first and second cell processing steps in at least one of the first and second modules. Each of the first and second cell processing steps may include measuring a parameter and determining if a pre-selected condition has been met by the measured parameter. At least one of the first and second cell processing steps may include automatically transferring the cartridge between the first instrument bay and the sterile liquid transfer instrument by the robot in accordance with the pre-programmed workflow. The method may further comprise automatically continuing, modifying, or stopping the workflow based upon at least one determination.

The pre-selected condition may comprise a cell concentration value, and the cell concentration value comprises a CD4+ concentration value greater than about 90%. The pre-selected condition may comprise a cell concentration value, and the cell concentration value may comprise a CD8+ concentration value greater than about 90%. The pre-selected condition may comprise a cell recovery value, and the cell recovery value may comprise a CD4+ recovery value greater than about 30%. The pre-selected condition may comprise a cell recovery value, and the cell recovery value may comprise a CD8+ recovery value greater than about 30%. The pre-selected condition may comprise a cell diameter value, and the cell diameter value may be greater than about 10 micrometers. The pre-selected condition may comprise a cell viability value, and the cell viability value may be greater than about 70%. The pre-selected condition may comprise a glucose value, and the glucose value may be greater than about 2 g/L. The pre-selected condition may comprise a lactate value, and the lactate value may be less than about 2 g/L. The pre-selected condition may comprise a chimeric antigen receptor expression value, and the chimeric antigen receptor expression value may be greater than about 10%.

In some variations, a method of growing cells in a closed, automated workcell may comprise providing a first cartridge, which may include a first bioreactor for housing and culturing cells, automatically culturing the cells in the first bioreactor, automatically transferring a sample of the cells from the first bioreactor to an analytical instrument of a sampling system within the automated workcell, automatically using the analytical instrument to determine a parameter of the cells within the first bioreactor, and upon a determination that the parameter has reached a threshold value, automatically transferring the cells from the first bioreactor to a second bioreactor to grow additional cells. The second bioreactor may be within the first cartridge. In some variations, the second bioreactor may be within a second cartridge. Transferring the sample of cells from the first bioreactor to the analytical instrument of the sampling system may be performed by using a sterile liquid transfer device and a robot. The analytical instrument may determine the parameter at a predetermined time interval. Culturing the cells in the first bioreactor may comprise one or more of cell enrichment, cell washing, cell incubation, cell selection, cell activation, cell transduction, and cell transfection. The method may further comprise retaining the cells in the first bioreactor upon a determination that the parameter has not reached the threshold value.

In some variations, a method of optimizing cell growth in a closed, automated workcell may comprise providing a first cartridge including a first bioreactor for housing and culturing cells, culturing the cells in the first bioreactor, transferring a sample of the cells from the first bioreactor to an analytical instrument for measuring a parameter, and based on the measured parameter. (i) maintaining existing conditions in the first bioreactor: (ii) performing a perfusion with 25% or more media exchange; or (iii) transferring cells from the first bioreactor to a second bioreactor.

In some variations, a method of culturing cells in a closed, automated workcell may comprise providing a first cartridge including a first bioreactor for housing and culturing cells. The method may further comprise providing a transfection reagent to the cells via a first sterile liquid transfer device to transfect the cells within the first bioreactor of the first cartridge and providing a transduction reagent to the cells via a second sterile liquid transfer device to transduce the cells within the first bioreactor of the first cartridge. Providing the transfection reagent may occur at a first predetermined time interval. Providing the transduction reagent may occur at a second predetermined time interval. The method may also comprise culturing the cells in the first bioreactor, transferring a sample of the cells from the first bioreactor to a sampling system within the automated workcell, using the sampling system to determine a parameter within the first bioreactor, and, upon a determination that the parameter has reached a threshold value, transferring the cells from the first bioreactor to a second bioreactor to spawn additional cells. Transferring the sample of the cells may occur at a third predetermined time interval.

Additional embodiments, features, and advantages of the invention will be apparent from the following detailed description and through practice of the invention.

### BRIEF DESCRIPTION OF THE DRAWINGS

FIG. 1A is a block diagram of an illustrative variation of a cell processing system.

FIG. 1B is a block diagram of a cartridge that may be provided to the cell processing system of FIG. 1A.

FIG. 2A is a rendering of an illustrative variation of a cell processing system. FIG. 2B is a perspective view of the cell processing system of FIG. 2A. FIG. 2C is a perspective view of the cell processing system of FIG. 2A and a cartridge. FIG. 2D is a rendering of an alternative variation of a cell processing system. FIG. 2E is a rendering of yet another variation of a cell processing system.

FIG. 3 is a rendering of an alternative variation of a cell processing system.

FIG. 4A is a flowchart of an illustrative variation of a conditional execution method.

FIG. 4B is a flowchart of an alternative variation of a conditional execution method.

FIG. 5A is a flowchart of an alternative variation of a conditional execution method.

FIG. 5B is a flowchart of yet another variation of a conditional execution method. FIG. 5C is a flowchart of an illustrative variation of a cell spawning method. FIG. 5D is a flowchart of an alternative variation of a cell spawning method. FIG. 5E is a flowchart of yet another variation of a cell spawning method.

### DETAILED DESCRIPTION

Disclosed herein are devices, systems, and methods for conditional execution of cell processing workflows in an automated cell processing system. Generally, the systems and devices described herein may perform methods of automatically executing a cell processing workflow, which may increase operational efficiency (e.g., reduce delays), reduce and/or eliminate manual intervention (e.g., labor), optimize workspace usage, and/or increase throughput of cell processing byproducts. The execution of the cell processing workflow may be conditional, such that a measurement may be compared to a pre-defined condition to determine whether the pre-defined condition has been met and the workflow may be continued or modified accordingly. The conditional execution of the workflow may be performed automatically, which is to say that human intervention may not be required at any step of the workflow. The workflow may include cell processing steps required to manufacture and/or evaluate cells. For example, the cell processing steps may include enrichment, incubation, selection (e.g., isolating), activation, purification, expansion, perfusion (e.g., dilution), washing, transduction, transfection, sampling, analyzing, and harvesting. The workcell may perform more than one workflow at a given time, which may further increase the operational efficiency of the cell processing methods described herein. For example, two or more cartridges may be collocated within the workcell, such that the workcell may independently perform cell processing steps associated with cell solutions contained in each cartridge. A robot may be configured to move each cartridge to facilitate the workflows described herein. In this way, multiple workflows may be performed automatically by the workcell, which may reduce operator intervention and increase throughput of cell product manufacturing.

### 1. Cell Processing System

The cell processing systems described herein may be configured to perform one or more cell processing steps in a workcell. The workcell may comprise a closed, automated environment, which may be configured to maintain a sterile environment. The workcell may receive a cartridge and perform one or more cell processing steps on cells in a cell solution contained within the cartridge. For example, the cell processing system may comprise a workcell comprising a plurality of instruments that may each be configured to independently perform one or more cell processing steps to the cells and/or cell solution, and a robot capable of moving the cartridge within the workcell (e.g., between one or more instruments). The robot and/or instruments may be configured to automatically operate such that operator assistance may not be required at any point during the workflow. For example, the robot may receive the cartridge and move the cartridge between locations (e.g., instruments, bays, storage vaults, feedthroughs) within the workcell according to a pre-programmed workflow; where each location may be associated with one or more cell processing steps. After performing one or more cell processing steps of the pre-programmed workflow, the workcell may be configured to transfer the cartridge out of the workcell (e.g., via the robot). Additionally or alternatively, at least a portion of the cell solution may be transferred (e.g., via a fluid transfer container) to a second cartridge.

The cell solution described herein may contain cells that may be processed for subsequent use in cell therapies. The cell solution may comprise cells (e.g., allogeneic cells) in a fluid, such as a media (e.g., cell culture media). The cell solution may contain cells from the same or different donors. Cells from the same donor may be split between one or more cartridges, such that separate cell processing steps may be performed on each of cartridge and increase the overall throughput of the cell processing system described herein. The cell solution may be transferred to the cartridge prior to loading the cartridge into the workcell, such as by operating personnel. In some variations, the cartridge may be empty when loaded into the workcell such that the workcell may transfer a cell solution to the cartridge. In some variations, the cells from two or more cartridges may be combined according to a pre-determined ratio, which may correspond to an intended therapeutic treatment for a patient.

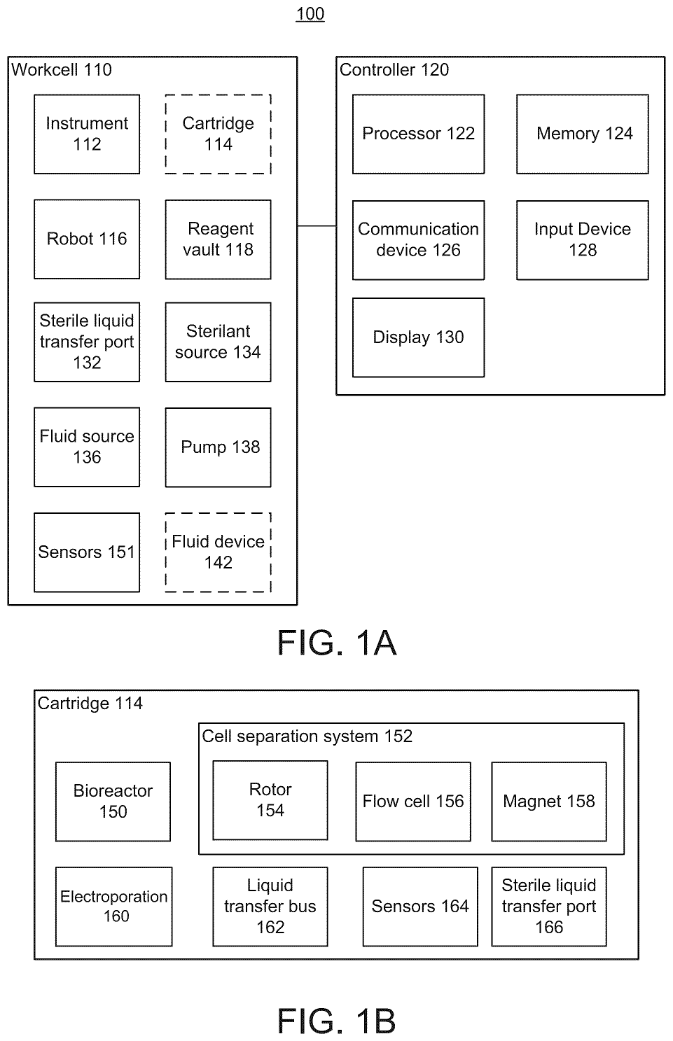

An illustrative cell processing system for use with the automated devices, systems, and methods is shown in FIG. 1A. Shown there is a block diagram of a cell processing system 100 comprising a workcell 110 and controller 120. The workcell 110 may comprise one or more of an instrument 112, a robot 116 (e.g., robotic arm), a reagent vault 118, a sterile liquid transfer port 132, a sterilant source 129, a fluid source 136, a pump 138, and a sensor(s) 151. A cartridge 114 and a fluid device 142, which may be provided outside of the workcell 110 and used within the workcell 110, are illustrated in dashed lines. In some variations, the fluid device 142 is a sterile liquid transfer device (SLTD). However, it should be appreciated that the fluid device 142 may be configured to transfer any fluid (which includes liquids), whether sterile or not. The controller 120 may comprise one or more of a processor 122, a memory 124, a communication device 126, an input device 128, and a display 130.

The workcell 110 may comprise a fully, or at least partially, enclosed housing inside which one or more cell processing steps may be performed in a fully, or at least partially, automated process. The cartridge 114 may be moved using the robot 116 to reduce manual labor in the cell processing steps, and fluid transfers into and out of the cartridge 114 may also be performed in a fully or partially automated process, as will be described in detail herein. For example, one or more fluids may be stored in a fluid device 142, such that the one or more fluids may be transferred to the cartridge 114 and/or removed from the cartridge 114 via the fluid device 142. In some variations, the fluid device 114 may be moved within the system 100 by the robot 116. Accordingly, the workcell 110 described herein advantageously enables the transfer of fluids in an automated and metered manner for automating cell therapy manufacturing.

The workcell 110 may facilitate fluid transfers and/or cartridge transfers. For example, in some variations, the robot 116 may be configured to move more than one cartridge 114 between different instruments to perform a predetermined sequence of cell processing steps (e.g., workflow). In this way, multiple cartridges 114 may be processed in parallel, as different steps of the cell processing workflow may be performed at the same time on different cartridges. In another example, a sterile liquid transfer port 132 may be coupled between two or more cartridges 114 to transfer a cell product and/or fluid between the cartridges 114. Furthermore, the sterile liquid transfer port 132 may be coupled between any set of fluid-carrying components of the system 100 (e.g., cartridge 114, reagent vault 118, fluid source 136, fluid device 142, etc.). For example, a first sterile liquid transfer port may be coupled between a first cartridge and a corresponding sterile liquid transfer port of a fluid device.

In some variations, a reagent vault 118 (or reagent vaults) may be used to store reagents, including but not limited to cell culture media, buffer, cytokines, proteins, enzymes, polynucleotides, reagents (e.g., transfection reagents, transduction reagents, microbeads), non-viral vectors, viral vectors, antibiotics, nutrients, cryoprotectants, solvents, cellular materials, and pharmaceutically acceptable excipients. Additionally, or alternatively, waste may be stored in the reagent vault, or within a fluid device within the reagent vault. In some variations, in-process samples extracted from one or more cartridges may be stored in the reagent vault, or in a fluid device within the reagent vault. The reagent vault may comprise one or more controlled temperature compartments (e.g., freezers, coolers, water baths, warming chambers, or others, at e.g., about −80° C., about −20° C., about 4° C., about 25° C., about 30° C., about 37° C., and about 42° C.). Temperatures in these compartments may be varied during the cell manufacturing process to heat or cool reagents.

In some variations, the reagents, waste, and/or extracted in-process samples, among others, may be stored within fluid devices 142 within the reagent vault 118. To this end, the fluid devices 142 may be transferred to a cartridge within the workcell or a cartridge may be moved by the robot 116 (or manually by an operator) to the reagent vault 118. The reagent vault 118 may interface with one or more sterile liquid transfer ports on the cartridge, and the reagent or material may be transferred from a fluid device 142 within the reagent vault into the cartridge. Optionally, fluid may be added or removed from the cartridge before, during, or after addition or removal of the reagent or material. In some variations, the instruments 112 of the workcell 110 may comprise a sterile liquid transfer instrument, similarly configured to transfer fluid into or out of the cartridge in an automated fashion. The sterile liquid transfer instrument may be stocked with reagents by, for example, a robot 116 that moves fluid devices 142 comprising the reagents from a workcell feedthrough or other location to the sterile liquid transfer instrument. In some variations, the robot 116 moves a fluid device(s) 142 from the reagent vault 118 to the sterile liquid transfer instrument. The reagent vault 118 may have automated doors to permit access by the robot 116 to the fluid device(s) 142 stored therein.

In some variations, the sensor(s) 151 of the workcell 110 may comprise optical sensors proximate to aspects of a sterile liquid transfer instrument. The sensor(s) 151 may be used during an automated fluid transfer procedure to aid in the controlled flow of fluids from the fluid device to another fluid device or to a cartridge. In particular, the optical sensors can be arranged with a view to windows of the fluid device to detect the presence or absence of fluid within fluid conduits of the fluid device. In this way, the controller 120 can deliver metered amounts of fluid from the fluid device to an adjoined fluid device or cartridge.

As illustrated in FIG. 1B, the cartridge 114 may be configured to contain (e.g., house) a cell solution. Accordingly, the cartridge 114 may comprise one or more of a bioreactor 150, a cell separation system 152, an electroporation module 160, a fluid transfer bus 162, a sensor(s) 164, and a sterile liquid transfer port 166, as described in more detail herein. The bioreactor 150 may be configured to contain the cell solution. The bioreactor 150 may further comprise a mixing chamber, in which the cell solution may be mixed with one or more reagents. In some variations, the cartridge 114 may comprise a first bioreactor and a second bioreactor. The first and second bioreactors may be fluidically isolated from one another, such that cell solutions may be contained in each bioreactor without mixing. The bioreactor 150 may comprise an impeller configured to rotate and thus increase pressure and/or flow of a cell solution contained therein.

The cartridge 114 may be configured to perform one or more cell processing steps. As shown, the cartridge 114 may comprise a cell separation system 152, which may be configured to perform a cell processing step comprising cell separation. Accordingly, the cell separation system 152 may comprise one or more of a rotor 154, flow cell 156, and magnet 158. In some variations, the magnet 158 may comprise one or more magnets and/or magnet arrays. For example, the cell separation system 152 may comprise a first magnet configured to magnetically rotate a rotor 154 and a second magnet (e.g., magnet array) that may each be configured to magnetically separate cells in the flow cell 156. In some variations, the cartridge 114 may have a reduced set of modules, which may facilitate faster and/or cheaper manufacturing of the cartridge 114. For example, the cartridge 114 may only have one or more modules corresponding to cell processing steps associated only with cell culturing and/or sampling. That is, in some variations, the cartridge 114 may include the bioreactor 150 and the fluid transfer bus 162. In another variations, the cartridge 114 may further comprise an elutriation module (not shown).

The cartridge 114 may be portable to facilitate automated and sterile cell processing. For example, the cartridge may be configured to be movable by a user from a location outside of the workcell 110 to a feedthrough thereof. In another example, the cartridge 114 may be movable by a robot from the feedthrough of the workcell 110 to any instrument therein. Generally, each of the instruments of the workcell 110 may interface with its respective module or modules on the cartridge. For example, an electroporation module 160 on the cartridge 114 may interface with the electroporation instrument to perform an electroporation step on the cell product—and may also interface with common components, such as components of a fluidic bus line (e.g., pumps, valves, sensors, etc.). In some variations, multiple cartridges may be used to process a single cell product through transfer of the cell product from one cartridge to another cartridge of the same or different type and/or by splitting cell product into more cartridges and/or pooling multiple cell products into fewer cartridges.

Various materials may be used to construct the cartridge 114, including metal, plastic, rubber, and/or glass, or combinations thereof. The cartridge, its components, and its housing may be molded, machined, extruded, 3D printed, or any combination thereof. The outer housing of the cartridge may comprise a material suitable to establish an additional boundary (e.g., a non-permeable material) that may further protect the sterility of the cell product.

Other suitable electroporation systems and devices are provided in, e.g., U.S. Patent Application No. 63/453,730, which is incorporated by reference herein.

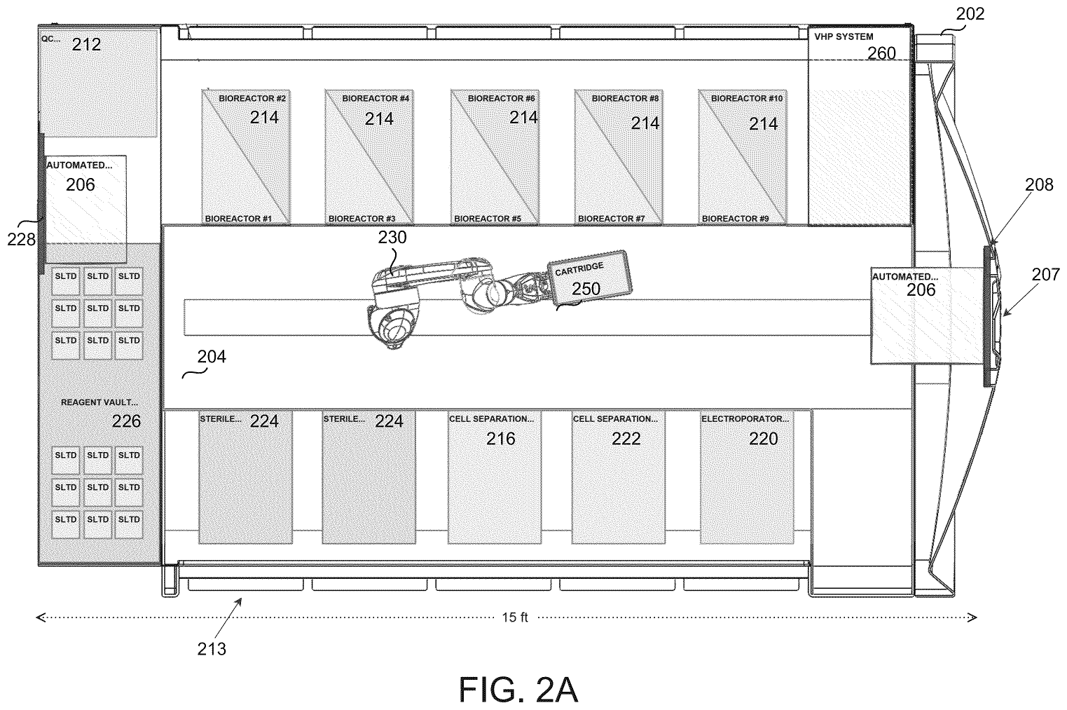

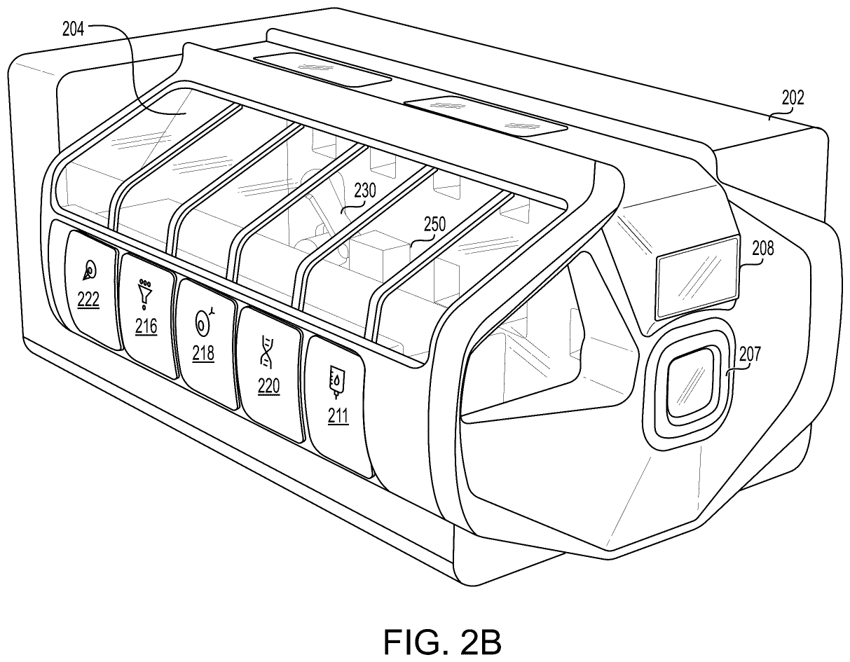

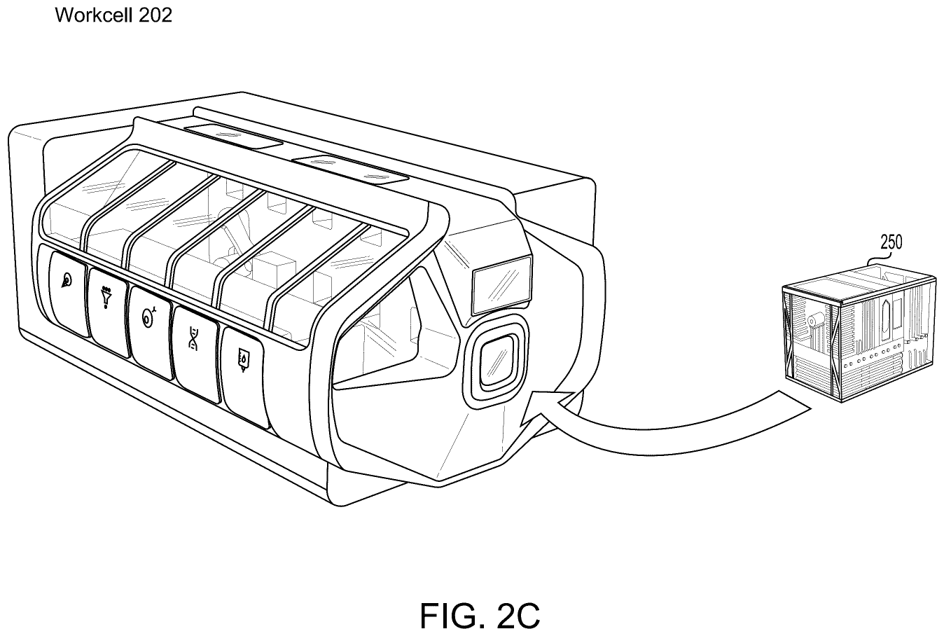

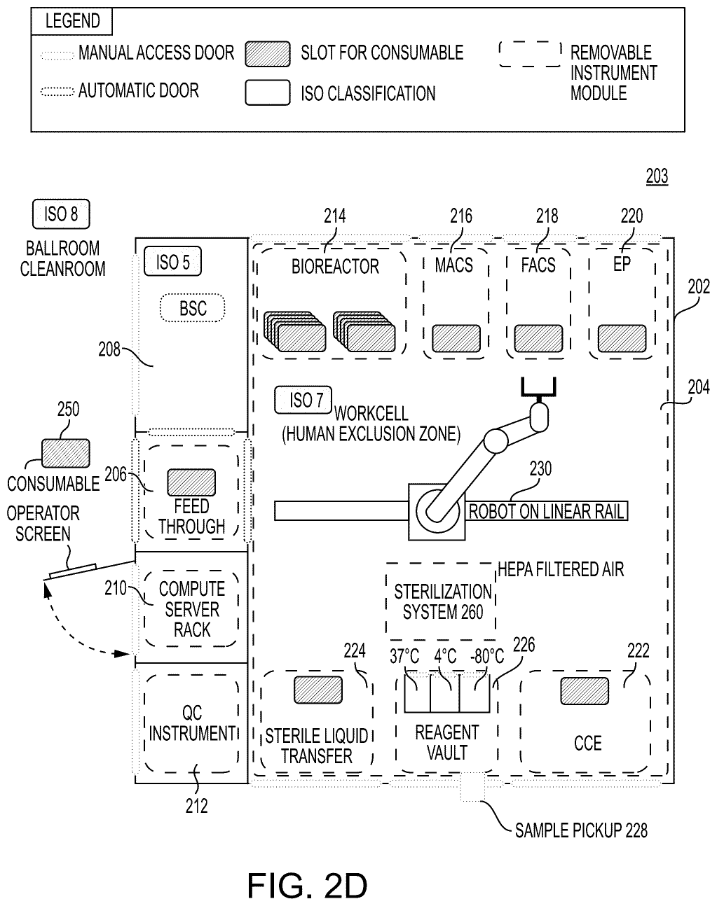

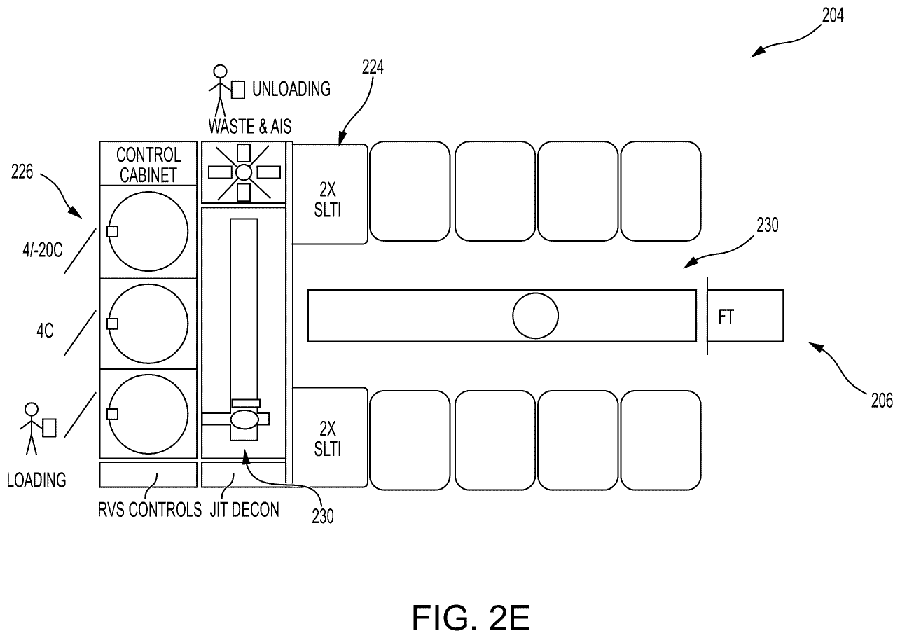

FIG. 2A shows an illustrative cell processing system for use with the devices, systems, and methods described herein. Shown there is workcell 202. The workcell may be divided into an interior zone 204 with a feedthrough 206 access, and quality control (QC) instrumentation 212. An air filtration inlet (not shown) may provide high-efficiency particulate air (HEPA) filtration to provide ISO7 or better air quality in the interior zone 204. In some embodiments, this air filtration may maintain sterile cell processing in an ISO8 or ISO9 manufacturing environment. The workcell 201 may also have an air filter on the air outlet to preserve the ISO rating of the room. Similar to the workcell described above in reference to FIG. 1A, the workcell 201 may further comprise, inside the interior zone 104, a bioreactor instrument 214, a cell selection instrument 216 (e.g., a magnetic separation instrument), an electroporation instrument 220, a counterflow centrifugation elutriation (CCE) instrument 222, a sterile liquid transfer instrument 224 (e.g., for facilitating automated fluid transfers), a reagent vault 226, and a sterilization system 260. The reagent vault 226 may be accessible by a user through a sample pickup port 228. A robot 230 (e.g., support arm, robotic arm, etc.) may be configured to move one or more cartridges 250 from any instrument to any other instrument, move one or more cartridges 250 to and from the reagent vault 226, and/or move one or more fluid devices between the reagent vault 226 and the sterile liquid transfer instrument 224. In some variations, the workcell 201 may comprise one or more moveable barriers 213 (e.g., access door) configured to facilitate access to one or more of the instruments in the workcell 201. FIG. 2B is a perspective view of a workcell 202 of a cell processing system. FIG. 2C is a perspective view of a cell processing system depicting a cartridge 250 introduced into a workcell 202. A plurality of cartridges may be inserted into the workcell 202 and undergo one or more cell processing steps in parallel.

FIG. 2D is a schematic illustration of an embodiment of a workcell 203. Workcell 203 may comprise an enclosure 202 having four walls, a base, and a roof. The workcell 203 may be divided into an interior zone 204 with a feedthrough 206 access, a biosafety cabinet (BSC) 208, compute server rack 210 (e.g., controller 120), and quality control (QC) instrumentation 212. An air filtration inlet (not shown) may provide high-efficiency particulate air (HEPA) filtration to provide ISO7 or better air quality in the interior zone 204. The workcell 203 may also have an air filter on the air outlet to preserve the ISO rating of the room. As with the workcell 202 described above in reference to FIGS. 2A-2C, workcell 203 may further comprise, inside the interior zone 204, an instrument 211 (e.g., disposed in a universal instrument bay), a bioreactor instrument 214, a cell selection instrument 216 (e.g., magnetic separation instrument, cell selection system), a cell sorting instrument 218 (e.g., FACS), an electroporation instrument 220, a counterflow centrifugation elutriation (CCE) instrument 222, and a sterile liquid transfer instrument 224, alongside a reagent vault 226 and a sterilization system 260 comprising one or more of a sterilant source, fluid source, and a pump. As will be described below, the sterilization system 260 may be connectable to a fluid device for sterilization of a sterile liquid transfer port during an automated fluid transfer process. The reagent vault 226 may be accessible through a sample pickup port 228. A robot 230 (e.g., support arm, robotic arm) may be configured to move one or more cartridges 250 from any instrument to any other instrument, move one or more cartridges 250) to and from the reagent vault 226, and/or move one or more fluid devices between the reagent vault 226 and the sterile liquid transfer instrument 224.

In some variations, a human operator may load one or more cartridges 250 into the feedthrough 206. The cartridges 250 may be pre-sterilized, or the feedthrough 206 may sterilize the cartridge 250 using ultraviolet radiation (UV) or chemical sterilizing agents provided as a spray or wash. The feedthrough 206 chamber may optionally be configured to automatically spray, wash, irradiate, or otherwise treat cartridges (e.g., with ethanol and/or isopropyl alcohol solutions) to maintain sterility of the interior zone 204 (e.g., ISO 7 or better) or the biosafety cabinet 208 (e.g., ISO 5 or better). The cartridge 250 may be passed to the biosafety cabinet 208, where input cell product is provided and loaded to the cartridge 250. The user may then move the cartridge 250 back to the feedthrough 206 and initiate automated cell processing using a computer processor in the computer server rack 210 (e.g., controller 120). The robot 230 may be configured to move the cartridge 250 in a predefined sequence to a plurality of instruments and stations, with the components of the workcell 203 being controlled by the computer processor of the computer server rack 210.

FIG. 2E shows an illustrative cell processing system for use with the devices, systems, and methods described herein. In some variations, a workcell 204 includes robot(s) 230 of a material handling system of the workcell 204. In some variations, a human operator may load one or more cartridges into the feedthrough 206. The workcell 204 may be divided into an interior zone with a feedthrough 206 access, a controls cabinet, reagent vault system (RVS) controls, just in time (JIT) decontamination, and an analytical instrument system (AIS). As with the workcell described above, workcell 204 may further comprise a plurality of instruments (e.g., disposed in a universal instrument bay), such as a bioreactor instrument, a cell selection instrument (e.g., magnetic separation instrument, cell selection system), a cell sorting instrument (e.g., FACS), an electroporation instrument, a counterflow centrifugation elutriation (CCE) instrument, and a sterile liquid transfer instrument, alongside a reagent vault 226 and a sterilization system comprising one or more of a sterilant source, fluid source, and a pump. The sterilization system may be connectable to a fluid device for sterilization of a sterile liquid transfer port during an automated fluid transfer process. The reagent vault 226 may be accessible through a sample pickup port during loading and/or unloading. A robot 230 (e.g., support arm, robotic arm) may be configured to move one or more cartridges from any instrument to any other instrument, move one or more cartridges to and from the reagent vault 226, move one or more cartridges to and from a sterile liquid transfer instrument 224, and/or move one or more fluid devices between the reagent vault 226 and the sterile liquid transfer instrument 224.

Other suitable reagent vault systems are provided in, e.g., U.S. Patent Application No. 63/470,381, which is incorporated by reference herein. Other suitable sterile liquid transfer instruments and systems are provided in, e.g., U.S. Patent Application No. 63/524,596, which is incorporated by reference herein. Other suitable sterile liquid transfer devices are provided in, e.g., U.S. Patent Application No. 63/456,388, which is incorporated by reference herein.

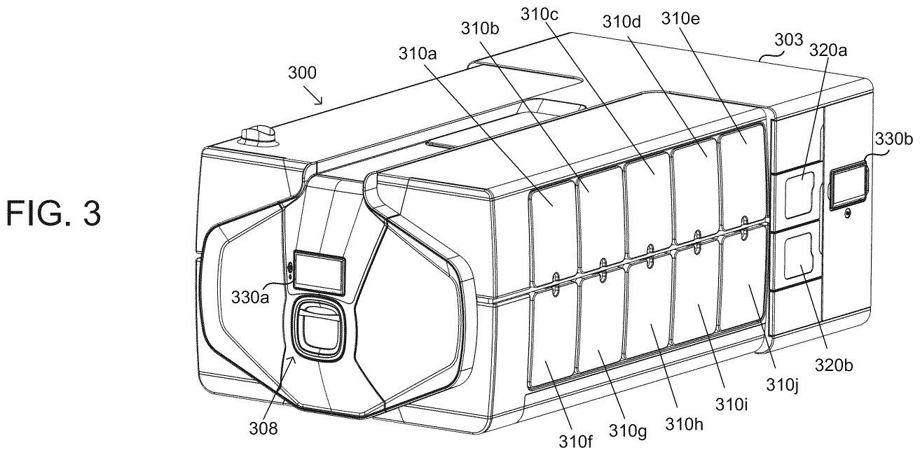

FIG. 3 shows an illustrative cell processing system comprising one or more instruments bays that may each be configured to perform one or more cell processing steps. As shown, a workcell 300 may comprise a plurality of instrument bays 310a-310j. Each of the instrument bays 310a-310j may comprise one or more of a bioreactor instrument, a cell selection instrument (e.g., magnetic separation instrument, cell selection system), a cell sorting instrument (e.g., FACS), an electroporation instrument, a spinoculation instrument, and a counterflow centrifugation elutriation (CCE) instrument. In this way, the instrument bays 310a-310j may each be referred to as a multi-functional instrument bay. In some variations, the instrument bay 310a may be referred to as a first instrument bay, the instrument bay 310b may be referred to as a second instrument bay, and so on. Each of the instrument bays 310a-310j may be configured to receive a cartridge (not shown). The cartridge may be loaded into each instrument bay via a robot, similar to the robots described in reference to FIGS. 2A-2E. The workcell 300 may further comprise a plurality of sterile liquid transfer instruments 320a-320b. Each of the sterile liquid transfer instruments 320a-320b may be configured to receive the cartridge (not shown) comprising one or more modules that engage or interact with the different instruments of the instrument bays. The sterile liquid transfer instruments 320a-320b may function similarly to the descriptions provided previously in reference to element 224 shown in FIGS. 2A-2E. In this way, the cartridge may be transferred between any instrument bay 310a-310j and any sterile liquid transfer instrument 320a-320b, in accordance with a pre-programmed workflow and one or more cell processing steps thereof.

The functionality of the workcell 300 may be controlled by one or more controllers. As shown, the workcell 300 may comprise controllers 330a-330b. Each controller 330a-300b may be coupled to an external surface of the workcell 300, such that a user may utilize each controller 330a-330b without entering the interior zone of the workcell 300. In this way, one or more workflows may continue uninterrupted and/or the sterile environment therein may be maintained. The controller 330a may comprise a first controller, and may be coupled to an external surface of a proximal portion of the workcell 300. The controller 330b may comprise a second controller, and may be coupled to an external surface of a distal portion of the workcell 300. The controllers 330a-330b may be located such that a user may minimize movement and thus increase operational efficiency of the cell processing described herein. The controllers 330-330b may be used simultaneously. In this way, multiple users may engage with the workcell 300 to adjust and/or observe one or more workflows occurring therein. For example, each of the controllers 330a-330b may be used to pre-program one or more of a cell processing step, condition, and workflow.

Other suitable cell processing systems and aspects thereof are provided in, e.g., U.S. patent application Ser. No. 17/198,134, published as U.S. Patent Publication No. 2021/0283565, and U.S. patent application Ser. No. 29/898,923, each of which is incorporated by reference herein.

### A. Workcell

i. Controller

The workcell described herein may comprise a controller configured to control one or more functions of the workcell, such as a robot configured to automatically execute a pre-programmed workflow. The controller may be configured for use by a user, such that the user may pre-program one or more of a cell processing step, condition, and/or workflow. In this way, the controller may be configured for conditional execution of at least one automated cell processing workflow. The controller may be in communication with one or more instruments configured to perform one or more cell processing systems. The controller may also be in communication with a cell sampling system configured to perform one or more measurements of a cell solution. The controller may also be configured to display a status associated with the workflow, including one or more measurements associated with one or more cell processing steps, the location of any cartridge and/or fluid device, the location of any robot, and environmental conditions (e.g., temperature, pressure, humidity). The controller may be further configured to generate an alert to the user, which may occur if at least one or more conditions are not met by one or more measurements. The alert may be an alarm and/or a notification on the display of the controller.

With reference now back to FIG. 1, the cell processing systems described herein may comprise a controller 130 (e.g., computing device). The controller 130 may comprise one or more of a processor 132, memory 134, communication device 136, input device 138, and display 140. The controller 130 may be configured to control (e.g., operate) any component or setting within the workcell 102. The controller 130 may comprise a plurality of devices. For example, the workcell 102 may enclose one or more components of the controller 130 (e.g., processor 132, memory 134, communication device 136) while one or more components of the controller 130 may be provided remotely to the workcell 102 (e.g., input device 138, display 140).

The processor (e.g., processor 132) described here may process data and/or other signals to control one or more components of the system. The processor may be configured to receive, process, compile, compute, store, access, read, write, and/or transmit data and/or other signals. Additionally, or alternatively, the processor may be configured to control one or more components of a device (e.g., console, touchscreen, personal computer, laptop, tablet, server). In some variations, the processor 132 may be configured to access or receive data and/or other signals from one or more of workcell 102, server, controller 130, and a storage medium (e.g., memory, flash drive, memory card, database). The processor 132 may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data transfer), and/or central processing units (CPU). The processor may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including structured text, typescript, C, C++, C#, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

The cell processing systems and devices described here may include a memory (e.g., memory 134) configured to store data and/or information. In some variations, the memory may include one or more of a random-access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with the device, such as image processing, image display, sensor data, data and/or signal transmission, data and/or signal reception, and/or communication. In some variations, a computer storage product with a non-transitory computer-readable medium (also may be referred to as a non-transitory processor-readable medium) having instructions or computer code thereon for performing various computer-implemented operations is used. In these variations, the computer-readable medium (or processor-readable medium) is non-transitory in the sense that it does not include transitory propagating signals per se (e.g., a propagating electromagnetic wave carrying information on a transmission medium such as space or a cable). The computer code (also may be referred to as code or algorithm) may be those designed and constructed for the specific purpose or purposes. The memory may be configured to store any received data and/or data generated by the controller and/or workcell. In some variations, the memory may be configured to store data temporarily or permanently.

In some variations, an input device 138, for example, may comprise or be coupled to a display. The input device may be any suitable device that is capable of receiving input from a user, for example, a keyboard, buttons, touch screen, etc. The input device may include at least one switch configured to generate a user input. For example, an input device may include a touch surface for a user to provide input (e.g., finger contact to the touch surface) corresponding to a user input. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In embodiments of an input device including at least one switch, a switch may have, for example, at least one of a button (e.g., hard key, soft key), touch surface, keyboard, analog stick (e.g., joystick), directional pad, mouse, railball, jog dial, step switch, rocker switch, pointer device (e.g., stylus), motion sensor, image sensor, and microphone. A motion sensor may receive user movement data from an optical sensor and classify a user gesture as a user input. A microphone may receive audio data and recognize a user voice as a user input.

In some variations, the cell processing system may optionally include one or more output devices in addition to the display, such as, for example, an audio device and/or a haptic device. An audio device may audibly output any system data, alarms, and/or notifications. For example, the audio device may output an audible alarm when a malfunction is detected. In some variations, an audio device may include at least one of a speaker, a piezoelectric audio device, a magnetostrictive speaker, and/or a digital speaker. In some variations, a user may communicate with other users using the audio device and a communication channel. For example, a user may form an audio communication channel (e.g., VOIP call). The haptic device may be configured to provide additional sensory output (e.g., force feedback) to the user. For example, the haptic device may generate a tactile response (e.g., vibration) to confirm user input to an input device (e.g., touch surface). As another example, haptic feedback may notify that user input is overridden by the processor.

In some variations, the controller may include a communication device (e.g., communication device 136) configured to communicate with another controller and one or more databases. The communication device may be configured to connect the controller to another system (e.g., Internet, remote server, database, workcell) by wired or wireless connection. The system may be in communication with other devices via one or more wired and/or wireless networks. In some variations, the communication device may include a radiofrequency receiver, transmitter, and/or optical (e.g., infrared) receiver and transmitter configured to communicate with one or more devices and/or networks. The communication device may communicate by wires and/or wirelessly.

Image data may be output on a display (e.g., display 140) of a cell processing system. The display may include at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

In some variations, as indicated above, a GUI may be configured for designing a process and monitoring a product. For example, the GUI may be a process design home page. The GUI may indicate that no processes have been selected or loaded. A create icon (e.g., “Create a Process”) may be selectable for a user to begin a process design process. In some variations, one or more of the GUIs described herein may include a search bar.

ii. Analytical Instruments

The cell processing systems described herein may comprise one or more analytical instruments configured to measure one or more parameters associated with cell solutions. In some variations, the analytical instrument may be integrated into the workcell (e.g., online) described previously and/or may be external to the workcell (e.g., offline). The analytical instruments, either online or offline, may be configured to perform one or more measurements associated with any cell processing step described herein. The measurements (e.g., measured parameters) obtained via the analytical instruments may be communicated to the controllers described previously. The controllers may compare the measured parameter to a pre-defined (e.g., pre-programmed, pre-selected) condition, such that the workflow may be continued, modified, or stopped based on the comparison (e.g., determination).

The cell processing system described herein may be configured to sample a portion of a cell solution, such that analysis may be performed on the sample before, during, and/or after one or more cell processing steps may be performed on the remaining cell solution. For example, a cartridge may be transferred (e.g., from an instrument bay or feedthrough) to a sampling instrument (e.g., a sterile liquid transfer instrument), where a sample of cell solution contained within the cartridge (e.g., in a bioreactor thereof) may be transferred to the sampling instrument. The fluid transfer may occur via one or more fluid paths (e.g., tubing, piping, conduits) between the cartridge and the sampling instrument. The transferred sample of the cell solution may correspond to a volume sufficient to accurately quantify at least one parameter associated with the cell solution (e.g., about 0.1 L to about 1 L). The sampling instrument may subsequently transfer the sample of cell solution to a portable fluid device (e.g., an SLTD). The portable fluid device may be moved by the robot described previously to facilitate offline or online analysis. In some variations, the sampling instrument (e.g., the sterile liquid transfer instrument) and the analytical instrument (e.g., one or more of the online and offline analytical instruments) may define a sampling system. In some variations, the sampling system may be configured to perform one or more measurements without sampling a portion of the cell solution. For example, the sampling system may comprise an analytical instrument configured to measure the cell solution while the cell solution may be housed in the cartridge. In such a variation, the analytical instrument may comprise one or more of an optical sensor, a pressure sensor, a scale, and a temperature probe.

Online sampling and analysis of a cell solution may be performed automatically, which may facilitate continuous (e.g., 24 hours a day, 7 days a week) cell processing. For example, the SLTD containing the sample of cell solution may be moved via the robot to an internal (e.g., integrated) analytical instrument within the workcell, such as an analytical instrument system (AIS). The AIS may be configured to receive the cell solution sample from the SLTD and measure one or more parameters of the cell solution. In another variation, online analysis may be performed on a cell solution contained within the cartridge, such that the cartridge need not be transferred between an instrument bay to the sterile liquid transfer instrument for sampling. In this way, movement of the cartridge may be reduced or eliminated and the operational efficiency of the workcell increased accordingly. That is, one or more cell processing steps may be paused for a shorter period of time to allow the measurements to occur or, in some variations where measurements may be taken while a cell processing step is performed, may not be paused at all.

Offline sampling of a cell solution may be performed separately from the online sampling, such that the throughput of the overall cell processing system may be increased via simultaneous operations. For example, the SLTD containing the sample of cell solution may be moved via the robot to a feedthrough of the workcell, such that a user may remove the SLTD from the feedthrough and transfer the SLTD to an external analytical instrument (e.g., flow cytometer). In this way, the cartridge may remain in the workcell for further cell processing while the external analytical instrument measures one or more parameters. The external analytical instrument may be configured to measure the same parameter(s) provided previously for the internal analytical instrument. In instances where the internal and external analytical instruments measure parameters associated with samples from the same cell solution, the measured parameters may be cross-referenced for quality control purposes. In some variations, the external analytical instrument may be configured to measure one or more different parameters than the internal analytical instrument, including parameters indicative of gene modification such as a chimeric antigen receptor expression value and a transgenic T cell receptor expression value. In some variations, both online and offline sampling may be performed substantially simultaneously on samples from the same or different cell solutions. In this way, the throughput of the workcell may be increased via the usage of both the online and offline sampling instruments.

The workflow may be pre-programmed to measure the one or more parameters associated with one or more cell processing steps at pre-determined time intervals. In some variations, the measured parameter may be associated with the cells within the cell solution, such as one or more of a total cell count value, a target cell count value, a cell concentration value, a cell recovery value, a cell diameter value, and a cell viability value. In further variations, the measured parameter may be associated with the media of the cell solution, such as one or more of a glucose value, a lactate value, a dissolved oxygen value, and a pH value.

The total cell count value may correspond to, for example, an expansion step. The total cell count value may indicate the number of substantially all cells (e.g., one or more cell types) within the cell solution. The number of cells within a cell solution may increase as cell growth (e.g., spawning) occurs. Accordingly, the total cell count value may be useful in determining whether a number of cells should be added to, or removed from, the cell solution before proceeding to a subsequent cell processing step.

The target cell count value may correspond to, for example, a cell selection step. The measured target cell count value may indicate the number of target cells (e.g., cells intended to be bound to a reagent) in the cell solution. The number of target cells within a cell solution may vary as the cell solution may be processed (e.g., incubated, sorted, perfused, harvested). Accordingly, the target cell count value may be useful in determining the amount of a reagent that should be added to the cell solution.

The cell concentration value (also referred to as a volumetric cell density value or a cell purity value) may correspond to, for example, an enrichment, expansion, depletion, perfusion, and/or selection step. The cell concentration value may represent the number of cells of a given type (e.g., white blood cells (WBC), CD3+ cells, CD4+ cells, CD8+ cells) within a cell solution divided by the total volume of the cell solution. The total volume of the cell solution may be pre-determined (e.g., based on the maximum fluid volume of a fluid device) and/or may be measured by an analytical instrument. The concentration of cells of a given type may vary as the cell solution may be processed (e.g., sorted, perfused, harvested). Accordingly, the cell concentration value may be useful in determining, for example, the amount of a reagent that should be added to the cell solution, eligibility of the cells for cell harvesting, eligibility of the cells for seeding, or combination thereof.

The cell recovery value may correspond to, for example, a cell selection step. The measured cell recovery value may represent a percentage of the number of cells of a given type (e.g., T cells) at the end of the cell processing step divided by the number of cells of the given type at the beginning of the cell processing step. Accordingly, in some variations, determining the cell recovery value may comprise performing at least two measurements, with at least one measurement occurring at the beginning of a cell processing step and at least one measurement occurring at the end of the cell processing step. A number of cells may be removed and/or damaged during one or more cell processing steps. Accordingly, the cell recovery value may be useful in determining, for example, whether a sufficient number of cells remain after a cell processing step to proceed to a subsequent cell processing step.

The cell diameter value may correspond to, for example, an activation step. The cell diameter value may correspond to a dimension (e.g., diameter) of a given cell. Cell dimensions, such as the cell diameter, may increase as the cell grows in size in preparation for division. The measured cell diameter value may be a value averaged across multiple cells or, in some variations, may be a value of a single cell. Accordingly, the measured cell diameter value may be useful in determining whether cells are metabolically active (e.g., growing and/or dividing).

The cell viability value may correspond to, for example, an activation step. The cell viability value may represent the number of cells that may be suitable for subsequent cell growth. For example, the cell viability value may be calculated using a number of live cells divided by the total number of cells (e.g., both live and dead cells). Accordingly, the cell viability value may indicate the percentage of cells that are alive and thus suitable for continued processing. Similar to the cell diameter value, the measured cell viability value may be useful in determining, for example, whether cells are metabolically active.

The glucose value may correspond to, for example, a perfusion step. The glucose value may represent the amount of glucose within the cell solution. Glucose may be a nutrient consumed by cells during the cell growth process. Accordingly, the glucose value may be useful in determining, for example, whether additional glucose (e.g., via perfusion with fresh media) should be added to the cell solution to provide the nutrients necessary for cell growth.

The lactate value may correspond to, for example, a perfusion step. The lactate value may represent the amount of lactate within the cell solution. Lactate may be produced (e.g., expelled) by cells during the cell growth process. Accordingly, the lactate value may be useful in determining, for example, whether any lactate may be removed from the cell solution (e.g., via perfusion with fresh media) to minimize the amount of lactate and maximize the amount of glucose in the cell solution.

The dissolved oxygen (DO) value may correspond to, for example, a perfusion step. The DO value may represent the amount of oxygen dissolved within the cell solution. Dissolved oxygen may be consumed by cells during cell growth. Accordingly, the DO value may be useful in determining, for example, whether additional DO may be added to the cell solution to enable the desired cell growth rate.

The pH value may correspond to, for example, an expansion, depletion, and/or perfusion. The pH value may represent the average pH of the cell solution. The average pH of the cell solution may correspond to the growth rate of the cells therein. For example, a cell growth rate may be relatively low if the average pH is substantially alkaline or substantially acidic. In an exemplary variation, a stable pH of about 7 to about 8, such as about 7.4, may correspond to an optimized cell growth rate. Accordingly, the pH value may be useful in evaluating, for example, an expected cell growth rate within the cell solution.

The chimeric antigen receptor expression value may correspond to, for example, a transduction, electroporation, and/or harvesting step. The chimeric antigen receptor expression may indicate the number of the number of cells within a cell solution that may be genetically modified, such as by having a T-cell receptor knocked out and replaced by a chimeric antigen receptor (CAR). In some variations, a given cell may comprise more than one chimeric antigen receptor. Therefore, the chimeric antigen receptor expression value may correspond to the total number of chimeric antigen receptors. The chimeric antigen receptor expression value may be useful in determining, for example, the number of cells suitable for use in cell therapy and/or seeding.

The transgenic T-cell receptor expression value may correspond to, for example, a transduction, electroporation, and/or harvesting step. The transgenic T-cell receptor expression value may indicate the number of cells within a cell solution that may be genetically modified, such as by having a T-cell receptor knocked out and replaced by a transgenic T cell receptor that may be configured to target a specific disease (e.g., cancer). In some variations, a given cell may comprise more than one transgenic T-cell receptor. Therefore, the transgenic T-cell receptor expression value may correspond to the total number of chimeric antigen receptors. The transgenic T-cell receptor expression value may be useful in determining, for example, the quantity of cells suitable for use in cell therapy and/or seeding.

While the parameters included herein may be described as related to a specific cell processing step, any of the parameters may be used in any of the cell processing steps, including the workflow variations described in detail below.

### II. Methods of Conditional Execution

Generally, the systems and devices described herein may perform one or more methods of automatically executing a cell processing workflow, which may increase operational efficiency (e.g., reduce delays), reduce and/or eliminate manual intervention (e.g., labor), optimize workspace usage, and/or increase throughput of cell processing byproducts. The execution of the cell processing workflow may be conditional, such that a measurement may be compared to a pre-defined condition to determine whether the pre-defined condition has been met and the workflow may be continued, stopped, or modified accordingly. Such conditional execution of the workflow may be performed automatically, which is to say that human intervention may not be required at any step in the workflow. Alternatively, the conditional execution of the workflow may be modified by human intervention at various steps of the workflow; predetermined time intervals, and the like. In general, the workcell described herein may measure a first parameter associated with a cell product, compare the first parameter against a first pre-defined condition and, if the first pre-defined condition is met, the workcell may continue with a pre-defined response in accordance with a pre-programmed workflow. If the first pre-defined condition is not met by the first parameter, the workcell may continue with a different pre-defined response in accordance with the pre-programmed workflow.

The pre-defined response may comprise continuing, stopping, and/or modifying the workflow: The pre-defined response may be pre-programmed using a controller by a user. The pre-defined response may correspond to a cell processing step within the workflow. The cell processing steps that may be performed by the workcell described herein may include any process required to manufacture and/or evaluate cells. For example, the cell processing steps may include sampling, enrichment, incubation, activation, growing, selection (e.g., isolating), purification, expansion, perfusion (e.g., dilution), washing, transduction, transfection, and harvesting.

Other suitable automated cell processing systems and methods are provided in, e.g., U.S. Patent Application No. 63/427,720, which is incorporated by reference herein.

The workflow described herein may comprise one or more cell processing steps associated with sampling, such that a portion of the cell solution may be transferred to an analytical instrument for analysis of at least one parameter associated with the cell solution. The sampling (e.g., via the sterile liquid transfer instrument) and/or analysis (e.g., via the online and/or offline analytical instruments) may be performed before, during, and/or after any cell processing step described herein. Accordingly, the analysis may evaluate a parameter associated with a given cell processing step, which may determine whether a cell processing step within a workflow may be continued, stopped, or modified. For example, at least one measured parameter may be compared to a pre-defined (i.e., pre-selected, pre-determined, pre-programmed) condition, which may represent a threshold value for a given parameter. The results of the comparison may determine an outcome. In some variations, a determination that the measured parameter meets or exceeds the condition may result in the workflow continuing. A determination that the measured parameter does not meet or exceed the condition may result in repeating at least one cell processing step. The workflow (e.g., sequence of one or more cell processing steps), pre-defined condition, and/or outcome may be pre-programmed using a controller by a user.

Other suitable sampling systems and devices are provided in, e.g., U.S. Patent Application No. 63/465,129, which is incorporated by reference herein.

The workflow described herein may comprise one or more cell processing steps associated with cell enrichment (e.g., purification), which may separate target cells from a starting material such that the target cells reach a concentration level appropriate for additional cell processing. The starting material (e.g., a leukopak) may comprise a cell solution comprising one or more types of cells (e.g., allogeneic cells) in media. The cell solution may be a liquid (e.g., fresh) or a solid (e.g., frozen). The enrichment may be performed by an elutriation instrument, such as a counterflow centrifugal elutriator. In some variations, the elutriation instrument may be configured to separate white blood cells from other material (e.g., red blood cells, platelets) within the starting material. The white blood cells may then be transferred to one or more modules to perform subsequent cell processing steps (e.g., incubation, selection). In an exemplary variation, the starting material may be loaded into a cartridge, which may be subsequently loaded into the workcell described herein. The starting material may be transferred to an elutriation module of the cartridge. In some variations, the elutriation module may be external to the cartridge such that the starting material may be transferred via a sterile liquid transfer device from the cartridge to the elutriation module (e.g., elutriation instrument).

One or more parameters may be measured before, during, and/or after cell enrichment, including a cell concentration value. In some variations, the measured cell concentration value may comprise a white blood cell concentration value. The measured white blood cell concentration value may be compared to a pre-defined condition. In some variations, the pre-defined condition may be a white blood cell concentration value of about 10%, about 20%, about 30%, about 40%, about 50%, about 60%, about 70%, about 80%, or about 90%. The comparison may indicate that the measured cell concentration value may be less than, equal to, or greater than the pre-defined cell concentration value. The outcome of the comparison may comprise stopping, continuing, or modifying the cell enrichment process.

Other suitable elutriation systems and devices are provided in. e.g., U.S. Patent Application No. 63/464,386, which is incorporated by reference herein.

The workflow described herein may comprise one or more cell processing steps associated with cell incubation, which may facilitate cell selection by binding one or more reagents to cells within a cell solution. In an exemplary variation, a cell solution may be contained within a mixing chamber of the cartridge, such that one or more reagents may be transferred thereto. The reagent may be transferred to the mixing chamber via, for example, an SLTD and a robot. The reagent may be configured to attach to certain cell types, such as CD4+ and/or CD8+ cells, according to the pre-programmed workflow. In some variations, the incubation may be performed with activation reagents, transfection reagents, transduction reagents, or combination thereof (each of which is described in further detail below).

The cell incubation process may be performed for a pre-determined duration, such that a pre-determined efficacy may be achieved. For example, the pre-determined duration may be about 10) minutes to about 12 hours, about 15 minutes to about 2 hours, or about 30 minutes to about 1 hour, such as about 30 minutes. The pre-determined duration may correspond to the cell concentration, volume of cell solution, environmental conditions of the cartridge and/or mixing chamber, and/or a combination thereof. The incubation process may be performed when the cell solution may be maintained at a pre-determined temperature, which may correspond to the type of reagent added to cell solution. For example, a reagent configured to target CD8+ cells (e.g., CD8 microbeads) may optimally bind to CD8+ cells at a pre-determined temperature of about 4 degrees C. In another example, a reagent comprising a liquid nanoparticle (LNP) may optimally bind to the target cells at a pre-determined temperature of about 37 degrees C. Accordingly, the pre-determined temperature may be about 0) degrees C. to about 40 degrees C. about 35 degrees C. to about 39 degrees C. (including about 37 degrees C.), or about 2 degrees C. to about 8 degrees C. (including about 4 degrees C.).

One or more parameters may be measured before, during, and/or after cell incubation, including a target cell count value. In some variations, the measured target cell count value may correspond to the number of CD4+ and/or CD8+ cells in the cell solution. The measured target cell count value may be compared to a pre-defined condition. In some variations, the pre-defined condition may be a target cell count value of about 500E5 cells to about 500E9 cells, including about 500E6 cells, about 600E6 cells, about 700E6 cells, about 800E6 cells, or about 900E6 cells. The comparison may indicate that the measured target cell count value may be less than, equal to, or greater than the pre-defined target cell count value. The outcome of the comparison may comprise stopping, continuing, or modifying the cell incubation process.

The workflow described herein may comprise one or more cell processing steps associated with gene modification, which may alter the genealogical make-up of cells within a cell solution to provide a therapeutic benefit. Gene modification may comprise transduction, which may include introducing a transduction reagent (e.g., a lentiviral vector and/or a virus) to a cell solution. The transduction reagent may be configured to target a specific cell type. Accordingly, the quantity (e.g., volume) of the transduction reagent may correspond to a cell concentration value within the cell solution. For example, a relatively large quantity of the transduction reagent may be introduced to a cell solution with a relatively high cell concentration value, and a relatively small quantity of the transduction reagent may be introduced to a cell solution with a relatively low cell concentration value. In this way, the quantity of transduction reagents may be approximately proportional to the concentration of target cells in the cell solution. The transduction reagent may be used to introduce, for example, a chimeric antigen receptor (CAR) to cells within the cell solution. In another example, a lentiviral vector comprising Lenti-CD19 CAR (scFv-41BB-CD3ζ, CTL019) may be configured to target CD19+ cells.

In some variations, gene modification may comprise electroporation. Electroporation may be configured introduce gene editing molecules into cells to manipulate their gene expression and/or cellular functions. Electroporation may be combined with different types of gene editing reagents to achieve different desired outcomes.

In some variations, electroporation may be performed on a cell solution to knock-out T cell receptors from cells therein, which may otherwise be recognized as foreign by a patient's cells and thus may be rejected. For example, electroporating cells with plasmids containing a Cas9 enzyme and guide RNA molecules that target the T cell receptor gene may lead to knock-out of the endogenous T cell receptor proteins, which may be necessary in reducing off-tumor, graft-versus-host-disease for allogeneic cell therapy. In another example, electroporation may be combined with viral transduction. The electroporation may first knock-out T cell receptors so that transduction may subsequently introduce a chimeric antigen receptor. Other reagents with similar gene editing capabilities, when combined with electroporation, may include plasmid DNA, Transcription Activator-Like Effector Nucleasus (TALENs), and Zinc Finger Nucleasus (ZFNs).

In yet further variations, gene modification may comprise transfection, which may introduce a transfection reagent (e.g., a nucleic acid) by a nonviral method. Transfection may be configured to knock-out certain cell types that may be associated with eliciting immune responses (e.g., graft vs host disease) in subsequent patients. That is, transfection may decrease the likelihood that a cell therapy recipient rejects the cells developed by the cell processing described herein. Transfection may be performed by adding one more transfection reagents to the bioreactor containing the cell solution, such that the cells may be modified by the transfection reagents. The transfection reagent may comprise an LNP. In some variations, the LNP may be provided in combination with an activation reagent (e.g., via CRISPR).

One or more parameters may be measured before, during, and/or after gene modification, including one or more of a cell concentration value, a chimeric antigen receptor expression value, and a transgenic T-cell receptor expression value. In some variations, the cell concentration value may comprise a CD3+ (which may include CD4+, CD8+, and/or CD19+ cells) concentration value. The measured parameter value may be compared to a pre-defined condition. In some variations, the pre-defined condition may be a CD3+ concentration value of about 10%, about 20%, about 30%, about 40%, about 50%, about 60%, about 70%, about 80%, or about 90%. In further variations, the pre-defined condition may be a chimeric antigen receptor expression value may be about 10%, about 20%, about 30%, about 40%, about 50%, about 60%, about 70%, about 80%, or about 90%. In yet further variations, the pre-defined condition may be a transgenic T-cell receptor expression value of about 10%, about 20%, about 30%, about 40%, about 50%, about 60%, about 70%, about 80%, or about 90%. The comparison may indicate that the measured parameter value may be less than, equal to, or greater than the pre-defined parameter value. The outcome of the comparison may comprise stopping, continuing, or modifying the gene modification process.

The workflow described herein may comprise one or more cell processing steps associated with cell activation, which may increase the size of individual cells within the cell solution such that the cells may divide after reaching an appropriate size. The activation process may comprise adding one or more activation reagents configured to bind to cell proteins associated with cell growth. For example, the activation reagent may comprise an antibody. The antibody may be configured to bind to a main T cell receptor and one or more proteins of the T cell (e.g., CD28), which may activate the T cell. In some variations, the activation reagent may be magnetically conjugated beads. In other variations, the activation reagent may be reversibly bound reagents having low binding affinity. The low binding affinity may be useful to avoid T cell exhaustion. For example, persistent and/or chronic exposure to activation reagents and subsequent stimulation can lead to T cell exhaustion. T cell exhaustion may be a state of functional impairment characterized by a gradual loss of effector functions, decreased cytokine production, reduced proliferation, and/or an overall inability to effectively carry out a cell's normal immune response duties. Therefore, a reversible activation reagent may prevent T cell exhaustion.

One or more parameters may be measured before, during, and/or after cell activation, including one or more of a cell diameter value and a cell viability value. The measured parameter value may be compared to a pre-defined condition. In some variations, the pre-defined condition may be a cell diameter value of about 1 micrometer to about 20 micrometers, including about 5 micrometers, about 10 micrometers, or about 15 micrometers. In further variations, the pre-defined condition may be a cell viability value of about 10%, about 20%, about 30%, about 40%, about 50%, about 60%, about 70%, about 80%, or about 90%. The comparison may indicate that the measured parameter value may be less than, equal to, or greater than the pre-defined parameter value. The outcome of the comparison may comprise stopping, continuing, or modifying the cell activation process.

After adding one or more reagents to the cell solution, the workflow described herein may comprise one or more cell processing steps associated with cell washing, which may remove unbound reagents from the cell solution. For example, the cell solution may be washed such that any unbound CD4+ microbeads, CD8+ microbeads, and/or LNPs may be removed from the cell solution. In this way, the cell solution may predominantly comprise bound pairs of cells and reagents, which may undergo further cell processing.

The workflow described herein may comprise one or more cell processing steps associated with cell selection (e.g., isolating), which may separate specific cell types from other cells. For example, cell selection may be performed via magnetic separation using one or more reagents. The magnetic selection may be configured to select cells bound to a specific reagent (e.g., microbead). In an exemplary variation, the magnetic selection process may select bound pairs of CD4+ cells and CD4+ microbeads. In another variation, the magnetic selection process may select bound pairs of CD8+ cells and CD8+ microbeads. The selection process may select more than one type of cells, such as both of CD4+ and CD8+ cells. The selected cells may be configured to undergo further cell processing or, in some variations, may be removed from the workcell for use in cell therapies.

One or more parameters may be measured before, during, and/or after cell selection, including one or more of a cell concentration value, a cell recovery value, and a target cell count value. In some variations, the cell concentration value may comprise a CD4+ and/or CD8+ concentration value. In further variations, the cell recovery value may comprise a CD4+ and/or CD8+ recovery value. The measured parameter value may be compared to a pre-defined condition. In some variations, the pre-defined condition may be a CD4+ and/or CD8+ concentration value of about 10%, about 20%, about 30%, about 40%, about 50%, about 60%, about 70%, about 80%, or about 90%. In further variations, the pre-defined condition may be a CD4+ and/or CD8+ recovery value may be about 10%, about 20%, about 30%, about 40%, about 50%, about 60%, about 70%, about 80%, or about 90%. In yet further variations, the pre-defined condition may be a target cell count value of about 500E5 cells to about 500E9 cells, including about 500E6 cells, about 600E6 cells, about 700E6 cells, about 800E6 cells, or about 900E6 cells. The comparison may indicate that the measured parameter value may be less than, equal to, or greater than the pre-defined parameter value. The outcome of the comparison may comprise stopping, continuing, or modifying the cell selection process.

The workflow described herein may comprise one or more cell processing steps associated with cell expansion, which may increase the number of cells within the cell solution. Cell expansion may be performed in the bioreactor of the cartridge described herein. For example, the bioreactor may comprise an impeller configured to mix the cell solution contained therein. The rate of rotation of the impeller may be adjusted to increase or decrease the efficacy of the mixing. For example, increasing the rate of rotation may correspond to a greater increase in the number of cells. Conversely, decreasing the rate of rotation may correspond to a lower increase in the number of cells. Cell expansion may be performed for a pre-determined duration, which may correspond to a target cell count. For example, a relatively low target cell count may correspond to a relatively short pre-determined duration (e.g., about 30 minutes up to about 72 hours), and a relatively high target cell count may correspond to a relatively long pre-determined duration (e.g., about 72 hours to about 14 days). In some variations, cell expansion may be combined with one or more cell processing steps. For example, perfusion may be combined with cell expansion such that a volume of cell solution may be replaced while the impeller may be rotating.

One or more parameters may be measured before, during, and/or after cell expansion, including one or more of a cell concentration value and a total cell count value. In some variations, the cell concentration value may comprise a CD4+ and/or CD8+ concentration value, as described previously. The measured parameter value may be compared to a pre-defined condition. In some variations, the pre-defined condition may be a CD4+ and/or CD8+ concentration value, as described previously. In further variations, the total cell count value may be about 500E6 cells to about 500E9 cells, including about 500E6 cells, about 500E7 cells, about 500E8 cells, about 20E9 cells, or about 500E9 cells. The comparison may indicate that the measured parameter value may be less than, equal to, or greater than the pre-defined parameter value. The outcome of the comparison may comprise stopping, continuing, or modifying the cell expansion process.

The workflow described herein may comprise one or more cell processing steps associated with perfusion (e.g., dilution, demidepletion), which may replace a pre-determined volume of media within the cell solution. Perfusion may be useful in maintaining an amount of nutrients in the cell solution associated with a desired cell growth rate. For example, cells may consume glucose during the cell growth process, so a low level of glucose may reduce or prevent cell growth. Conversely, cells may expel (e.g., excrete) lactate during the cell growth process, so an excess of lactate may inhibit cell growth. Accordingly, the perfusion process may ensure the amount of glucose within the cell solution remains above a level sufficient to support the desired cell growth and/or the amount of lactate within the cell solution remains below a level sufficient to prevent inhibiting cell growth. In some variations, perfusion may be performed continuously such that a volume of media may be constantly being removed and new (e.g., fresh) media may be constantly being added. In further variations, perfusion may be performed at discrete time intervals, which may correspond to a pre-determined workflow. For example, the time intervals may correspond to a measured parameter meeting or exceeding a pre-defined condition. Cells within the cell solution may be retained within the bioreactor (e.g., via a filter), such that only the media may be exchanged. In some variations, the pre-determined volume of media exchanged (e.g., removed and replaced) may be about 10% to about 100%, including about 25%, about 50%, and about 75% of the original cell solution. The pre-determined volume may be based on a cell concentration value and/or the pre-defined workflow: In some variations, perfusion may be performed within a first bioreactor of the cartridge. In other variations, at least a portion of the cell solution may be transferred to a second bioreactor, such that perfusion may be performed in the second bioreactor.

One or more parameters may be measured before, during, and/or after perfusion, including one or more of a lactate value, a glucose value, a dissolved oxygen value, and a pH value. The measured parameter value may be compared to a pre-defined condition. In some variations, the pre-defined condition may be a lactate value of about 1 g/L to about 5 g/L, including about 2 g/L. In further variations, the pre-defined condition may be a glucose value of about 1 g/L to about 5 g/L, including about 2 g/L. In yet further variations, the pre-defined condition may be a dissolved oxygen value of about 1 mg/L to about 15 mg/L, about 3 mg/L to about 13 mg/L, or about 6 mg/L to about 11 mg/L. For example, in some variations, the dissolved oxygen value may be about 1 mg/L. 3 mg/L. 5 mg/L. 6 mg/L. 7 mg/L, 8 mg/L, 9 mg/L, 10 mg/L, or 11 mg/L. In still further variations, the pre-defined condition may be a pH value of about 6 to about 8, including about 7, about 7.3, about 7.4, about 7.5, or about 7.75. The comparison may indicate that the measured parameter value may be less than, equal to, or greater than the pre-defined parameter value. The outcome of the comparison may comprise stopping, continuing, or modifying the perfusion process.

The workflow described herein may comprise one or more cell processing steps associated with cell depletion, which may remove unmodified cells from the cell solution. Cell depletion may be configured to separate cells within a cell solution using one or more depletion reagents. For example, the depletion reagents (e.g., depletion microbeads) may be added to the cell solution, which may be stored in the bioreactor, such that the cells may be incubated with the depletion reagents. That is, the depletion reagents may be configured to bind to certain cells (e.g., target cells), which may mark the non-bonded cells for subsequent removal. In an exemplary variation, the depletion reagents may bind to CD3+ cells (which may include CD4+ and CD8+ cells) such that the bounded CD3+ may be separated from unmodified T cells. Accordingly, the unmodified T cells may be removed from the cell solution.

Cell depletion may be performed at a pre-determined temperature, which may correspond to a desired efficacy rate (e.g., percentage of CD3+ cells successfully bound to a microbead reagent). For example, a relatively higher pre-determined temperature may correspond to a higher efficacy rate. In some variations, the pre-determined temperature may be about 30 degrees C. to about 40 degrees C., including about 37 degrees C. In some variations, cell depletion may be configured to filter the cell solution via bulk depletion or fine depletion, which may refer to a depletion time period. For example, in bulk depletion, a quantity of depletion reagents may be incubated with the cell solution for a bulk depletion time period, which may be about 2 hours to about 4 hours. In fine depletion, a quantity of depletion reagents may be incubated with the cell solution for a fine depletion time period, which may be about 2 hours to about 6 hours. Accordingly, in some variations, the fine depletion time period may be greater than the bulk depletion time period, such that a greater portion of the depletion reagents may bind to the targeted cells and thereby increase the efficacy rate of the depletion process.

One or more parameters may be measured before, during, and/or after cell depletion, including one or more of a cell concentration value and a pH value. In some variations, the cell concentration value may comprise a CD4+ and/or CD8+ concentration value, as described previously. The measured parameter value may be compared to a pre-defined condition. The pre-defined conditions for the cell concentration value and/or pH value may be similar to the previous descriptions. The comparison may indicate that the measured parameter value may be less than, equal to, or greater than the pre-defined parameter value. The outcome of the comparison may comprise stopping, continuing, or modifying the cell depletion process.

The workflow described herein may comprise one or more cell processing steps associated with harvesting, which may result in cells suitable for cell therapies in one or more patients. Harvesting may comprise removing cells from the cell solution (e.g., separating target cells from media) and transferring the cells to one or more containers (e.g., SLTDs, cryopreservation bags). The removed cells may be configured for a pre-determined cell therapy in one or more patients. In some variations, the removed cells may be placed in a bioreactor of a cartridge (e.g., seeding), such that the cell spawning process described herein may be performed to generate additional cells. In further variations, the removed cells may be preserved (e.g., via cryopreservation) for later use in one or more of cell growing processes and cell therapies.

One or more parameters may be measured before, during, and/or after harvesting, including one or more of a total cell count value, a cell concentration value, and a chimeric antigen receptor expression value. The total cell count value, cell concentration value, and chimeric antigen receptor expression value may be similar to the descriptions provided previously. The measured parameter value may be compared to a pre-defined condition. In some variations, the pre-defined condition may be a CD4+ and/or CD8+ concentration value, as described previously. In further variations, the total cell count value may be about 500E8 cells to about 500E9 cells, including about 4E9 cells. The comparison may indicate that the measured parameter value may be less than, equal to, or greater than the pre-defined parameter value. The outcome of the comparison may comprise stopping, continuing, or modifying the harvesting process.

The workflow described herein may comprise one or more cell processing steps associated with cryopreservation, which may preserve a cell solution for subsequent processing at a later time. The cell solution to be preserved may have undergone one or more cell processing steps. Accordingly, the cell solution described herein may be undergo cryopreservation at any point in the workflow. The cryopreservation may be configured to preserve cells within the cell solution without damaging and/or killing cells therein. Cryopreservation may be performed by removing any volume of cell solution from the cartridge and/or workcell and transferring the cell solution to a container configured to withstand the environmental conditions (e.g., temperature, pressure, humidity) associated with cryopreservation. In an exemplary variation, the container may comprise a bag manufactured from a plastic suitable for extremely cold temperatures (e.g., less than about-190 degrees C.). Any cell solutions that previously undergone cryopreservation may be thawed and transferred back to the cartridge and/or workcell. For example, a cell solution that had undergone cell separation prior to cryopreservation, which may have separated CD4+ and/or CD8+ cells, may be transferred to a bioreactor within the cartridge and subsequently used for further cell processing.

The workflow may be configured for any cell process, including but not limited to manufacturing cells. In some variations, there may be a plurality of workflows being performed within a workcell simultaneously, where each of the plurality of workflows may have concurrent or staggered timing relative to each other. In this way, the workcell may be configured to optimize the total throughput of cell products (e.g., harvested cells) by efficiently performing portions of each of the plurality of workflows.

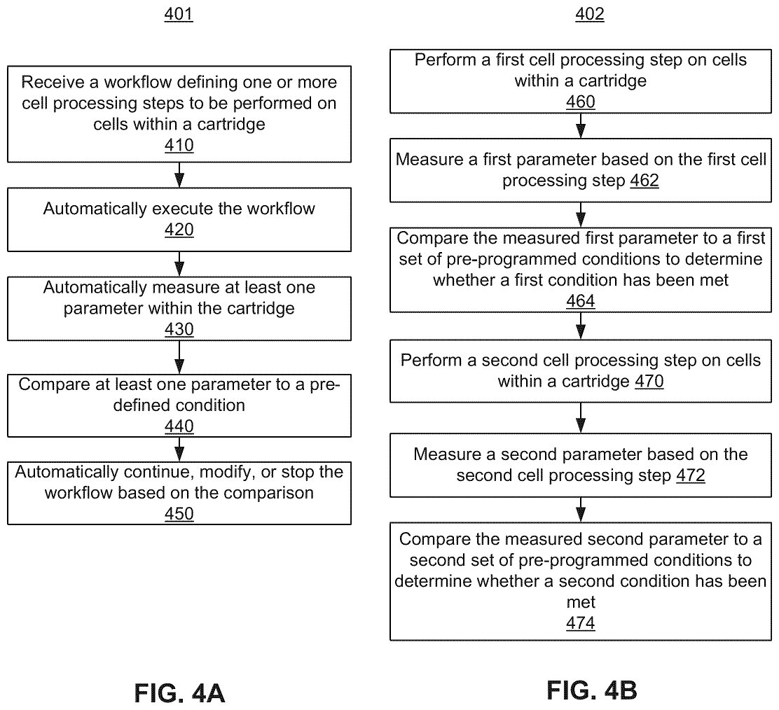

FIGS. 4A-4B provide flowcharts of illustrative methods of conditional execution of a workflow in a cell processing system as described herein. As shown in FIG. 4A, a method 401 may comprise receiving a workflow defining one or more cell processing steps to be performed on cells within a cartridge 410. The one or more cell processing steps may comprise one or more of enrichment, selection, activation, expansion, perfusion, sampling, and harvesting. The workflow may be pre-programmed using a controller by a user. The workflow may be associated with one or more cell processes, such as manufacturing cells. The method may further comprise automatically executing a pre-programmed workflow 420. The workflow may be executed by a workcell, which may comprise one or more of a robot, an instrument, a feedthrough, and a storage unit. At least one parameter within the cartridge may be automatically measured 430. The parameter may be measured by an online and/or offline analytical instrument. The measured parameter may be communicated to the controller. The at least one measured parameter may be compared to a pre-defined condition 440. The controller may be configured to perform the comparison. The pre-defined condition may be pre-defined using a controller by a user. The workflow may be continued, modified, or stopped based upon the comparison 450. For example, a comparison indicating that the measured parameter meets or exceeds the pre-defined condition may result in the workflow continuing, such that a subsequent cell processing step may be performed. A comparison indicating that the measured parameter does not meet the pre-defined condition may result in modifying the workflow, such as by repeating one or more cell processing steps, or stopping the workflow, such that an alert may be generated by the controller.

As shown in FIG. 4B, a method 402 may comprise performing more than one cell processing step and measuring more than one parameter. As illustrated, the method 402 may comprise performing a first cell processing step on cells within a cartridge 460. The first cell processing step may comprise any of the cell processing steps described herein. For example, the first cell processing step may comprise enrichment, which may be configured to separate target cells from other material in a cell solution. The method 402 may further comprise measuring a first parameter based on the first cell processing step 462. The first parameter may be measured by an online and/or offline analytical instrument. For example, the first parameter may comprise a cell concentration value, such as a white blood cell concentration value. The measured first parameter may be compared to a first set of preprogrammed conditions to determine whether a first condition has been met 464. For example, the first set of pre-programmed conditions may comprise a threshold value for the white blood cell concentration value. The comparison may be performed by the controller. If the comparison indicates that the measured parameter does not meet the threshold value, the first cell processing step may be repeated. The method 402 may comprise, upon a determination that the first condition has been met, performing a second cell processing step on the cells within the cartridge 470. The second cell processing step may comprise any of the cell processing steps described herein. For example, the second cell processing step may comprise cell activation. Cell activation may be performed by adding one or more reagents to the cell solution and allowing the one or more reagents to mix with the cell solution for a pre-determined duration. A second parameter based on the second cell processing step may be measured 472. The second parameter may be measured by an online and/or offline analytical instrument. For example, the second parameter may comprise a cell diameter value. The measured second parameter may be compared to a second set of preprogrammed conditions to determine whether a second condition has been met 474. In some variations, a user alert may be generated if either of the first or second conditions are not met. The workflow may be continued, modified, or stopped based on the comparison. In this way, the workflow may be conditionally executed based on the comparison of the measured parameter to a preprogrammed condition.

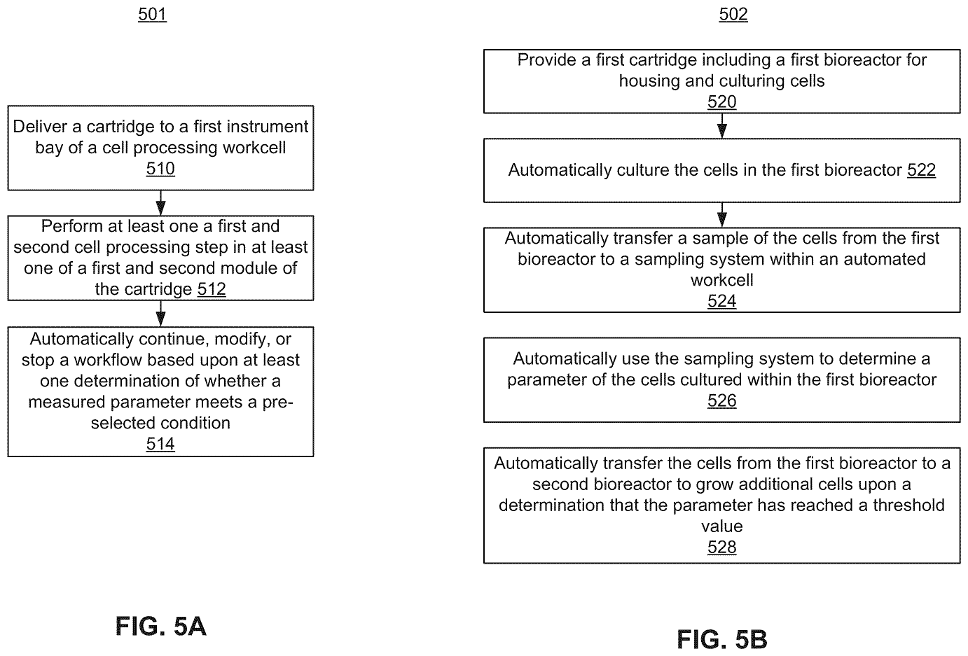

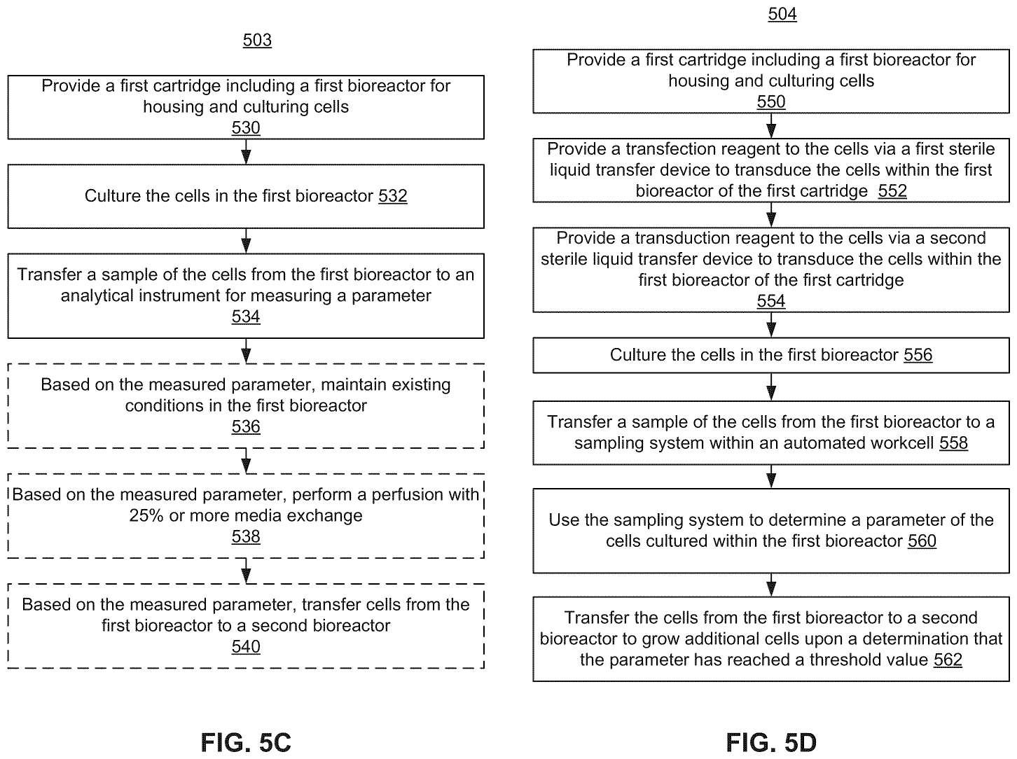

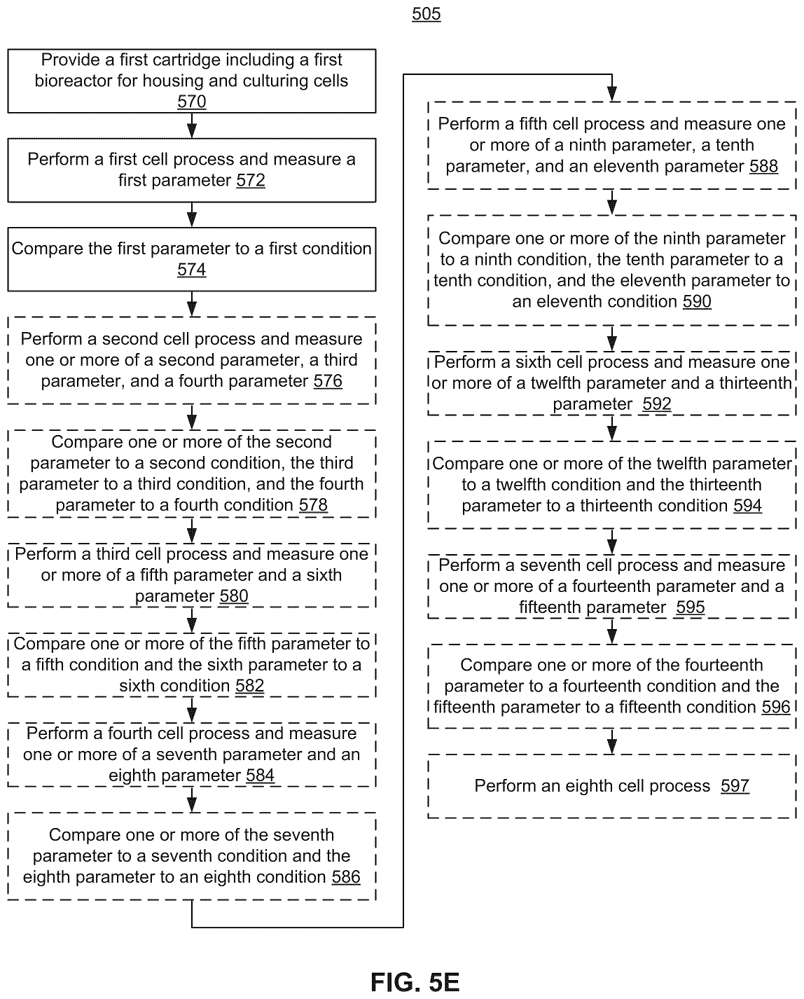

Conditional execution may be useful for manufacturing cells for use in cell therapies. Accordingly, FIGS. 5A-5D provide flowcharts of illustrative methods of manufacturing cells (e.g., cell spawning) via conditional execution of a workflow in a cell processing system as described herein. As shown in FIG. 5A, a method 501 may comprise delivering a cartridge to a first instrument bay of a cell processing workcell 510. The first instrument bay may comprise one or more instruments configured to perform one or more cell processing steps. The cartridge may be delivered to the first instrument bay via a robot. The cartridge may comprise at least a first module and a second module, and may contain cells in one or more of the first and second modules. The workcell may comprise the robot, a sterile liquid transfer instrument, and a controller comprising at least one pre-programmed workflow defining at least a first cell processing step and a second cell processing step. The method may further comprise performing at least one of the first and second cell processing steps in at least one of the first and second modules 512. The first and second cell processing steps may be independently selected from the group consisting of enrichment, selection, activation, expansion, perfusion, sampling, and harvesting.

Each of the first and second cell processing steps may include measuring a parameter and determining if a pre-selected condition has been met by the measured parameter. The measured parameter and associated pre-selected condition may comprise one or more of a total cell count value, a target cell count value, a cell concentration value, a cell recovery value, a cell diameter value, a cell viability value, a glucose value, a lactate value, and a chimeric antigen receptor expression value. For example, the pre-selected condition may comprise a total cell count value, and the total cell count value may be greater than a pre-selected value. The pre-selected condition may comprise a target cell count value, and the target cell count value may be greater than a pre-selected value. The pre-selected condition may comprise a cell concentration value, and the cell concentration value may be a white blood cell concentration value greater than about 80%. The pre-selected condition may comprise a cell concentration value, and the cell concentration value may comprise a CD4+ concentration value greater than about 90%. The pre-selected condition may comprise a cell concentration value, and the cell concentration value may comprise a CD8+ concentration value greater than about 90%. The pre-selected condition may comprise a cell recovery value, and the cell recovery value may comprise a CD4+ recovery value greater than about 30%. The pre-selected condition may comprise a cell recovery value, and the cell recovery value may comprise a CD8+ recovery value greater than about 30%. The pre-selected condition may comprise a cell diameter value, and the cell diameter value may be greater than about 10 micrometers. The pre-selected condition may comprise a cell viability value, and the cell viability value may be greater than about 70%. The pre-selected condition may comprise a glucose value, and the glucose value may be greater than about 2 g/L. The pre-selected condition may comprise a lactate value, and the lactate value may be less than about 2 g/L. The pre-selected condition may comprise a chimeric antigen receptor expression value, and the chimeric antigen receptor expression value may be greater than about 10%.

At least one of the first and second cell processing steps may include automatically transferring the cartridge between the first instrument bay and the sterile liquid transfer instrument by the robot in accordance with the pre-programmed workflow. The sterile liquid transfer instrument may be configured to transfer a sample of the cell solution to a fluid device, such as sterile liquid transfer device. The fluid device may be transferred to at least one analytical instrument via the robot. In some variations, the measured parameter may be measured by an analytical instrument of the first instrument bay. The method may further comprise automatically continuing, modifying, or stopping the workflow based upon at least one determination 514. For example, the first cell processing step may be repeated upon a determination that the measured parameter does not meet the pre-selected condition.

As shown in FIG. 5B, a method 502 may be directed to transferring cells between bioreactors to grow additional cells. As illustrated, the method 502 may comprise providing a first cartridge, which may include a first bioreactor for housing and culturing cells 520. The cells may comprise allogeneic cells. The method 502 may further comprise automatically culturing the cells in the first bioreactor 522. Culturing the cells in the first bioreactor may comprise one or more of cell enrichment, cell washing, cell incubation, cell selection, cell activation, cell transduction, and cell transfection. A sample of the cells from the first bioreactor may be automatically transferred to an analytical instrument of the sampling system within the automated workcell 524. Transferring the sample of cells from the first bioreactor to the analytical instrument of the sampling system may be performed by using a sterile liquid transfer device and a robot. The method 502 may also comprise automatically using the analytical instrument to determine a parameter within the first bioreactor 526. Upon a determination that the parameter has reached a threshold value, the method 502 may further comprise automatically transferring the cells from the first bioreactor to a second bioreactor to grow additional cells 528. The second bioreactor may be within the first cartridge. In some variations, the second bioreactor may be within a second cartridge. Upon a determination that the parameter has not reached a threshold value, the cells may be retained in the first bioreactor.

Transferring the sample of cells from the first bioreactor to an analytical instrument of the sampling system may be performed by using a sterile liquid transfer device and a robot. The sampling system may determine the parameter at a predetermined time interval. Culturing the cells in the first bioreactor may comprise one or more of cell enrichment, cell washing, cell incubation, cell selection, cell activation, cell transduction, and cell transfection. The method may further comprise retaining the cells in the first bioreactor upon a determination that the parameter has not reached the threshold value.

As shown in FIG. 5C, a method 503 may be directed to optimizing cell growth by maintaining or adjusting conditions within a bioreactor. As illustrated, the method 503 may comprise providing a first cartridge including a first bioreactor for housing and culturing cells 530. The cells may comprise allogeneic cells. The cells may be transferred to the first bioreactor before providing the first cartridge to the workcell. In some variations, an empty cartridge may be provided to the workcell and cells subsequently transferred to the cartridge via, for example, a robot. The method 503 may further comprise culturing the cells in the first bioreactor 532. Culturing the cells may comprise one or more of cell enrichment, cell washing, cell incubation, cell selection, cell activation, cell transduction, and cell transfection. Culturing the cells may be performed according to a pre-programmed workflow, which may be based on user input via the controller. Then, the method 503 may comprise transferring a sample of the cells from the first bioreactor to an analytical instrument for measuring a parameter 534. For example, the cartridge may be transferred to a sterile liquid transfer instrument within the workcell, such that the sample of the cells may be removed by the sterile liquid transfer instrument and transferred to a sterile liquid transfer device. The sterile liquid transfer device may be transferred to the analytical instrument via, for example, a robot. The analytical instrument may be internal or external to the workcell. Based on the measured parameter, the method 503 may comprise one or more optional steps. As illustrated, the method 503 may comprise maintaining the existing conditions in the first bioreactor 536. For example, the conditions may be maintained upon a determination that the measured parameter does not meet a pre-defined condition, such as a cell concentration value. Alternatively, the method 503 may comprise performing a perfusion with 25% or more media exchange. For example, perfusion may be performed upon a determination that the measured parameter meets or exceeds a pre-defined condition. The magnitude of media perfused may be proportional to the difference between the measured parameter and the pre-defined condition. In yet another alternative, the method 503 may comprise transferring cells from the first bioreactor to a second bioreactor. For example, the measured parameter may indicate that the cells may be used for seeding additional cell processes.

As shown in FIG. 5D, a method 504 may include providing a transfection reagent and a transduction agent to cells. As illustrated, the method 504 may comprise providing a first cartridge including a first bioreactor for housing and culturing cells 550, similar to the description in reference to FIG. 5C. The method 504 may further comprise providing a transfection reagent to the cells via a first sterile liquid transfer device to transfect the cells within the first bioreactor of the first cartridge 552. The transfection reagent may be provided at a first predetermined time interval. For example, the cartridge may be transferred to a sterile liquid transfer instrument. The first sterile liquid transfer device may be removed from a reagent vault by a robot and transferred to sterile liquid transfer instrument, such that the contents of the first sterile liquid transfer device may be transferred to the cartridge. Once the first sterile liquid transfer device may be emptied, the first sterile liquid transfer device may be transferred back to the reagent vault or, in some variations, may be transferred to a waste unit of the workcell for subsequent removal. Additionally, the method 504 may comprise providing a transduction reagent to the cells via a second sterile liquid transfer device to transduce the cells within the first bioreactor of the first cartridge 554. The transduction reagent may be provided at a second predetermined time interval. The second sterile liquid transfer device may be transferred in a similar manner as the first sterile liquid transfer device.

The method 504 may also comprise culturing the cells in the first bioreactor 556. Culturing the cells may comprise one or more of cell enrichment, cell washing, cell incubation, cell selection, and cell activation. Then, the method 504 may comprise transferring a sample of the cells from the first bioreactor to a sampling system within the automated workcell 558. Transferring the sample of the cells may occur at a third predetermined time interval. For example, the sampling system may comprise the sterile liquid transfer instrument and an analytical instrument. Accordingly, the sampling system may be used to determine a parameter within the first bioreactor 560. The sampling system, including the analytical instrument thereof, may be configured to measure one or more parameters associated with the cell solution. Upon a determination that the parameter has reached a threshold value, the method 504 may comprise transferring the cells from the first bioreactor to a second bioreactor to grow additional cells 562. The transfer from the first bioreactor to the second bioreactor may be referred to as seeding.

As shown in FIG. 5E, a method 505 may include a plurality of cell processes and comparing a plurality of measured parameters to a respective plurality of conditions according to a pre-programmed workflow. As illustrated, the method 505 may comprise providing a first cartridge including a first bioreactor for housing and culturing cells 570. The method 505 may further comprise performing a first cell process and measuring a first parameter 572. The first cell process may comprise enrichment. The first parameter may be measured at a first time interval. For example, the first parameter may be a cell concentration value, such as a white blood cell concentration value. The white blood cell concentration value may be measured by a flow cytometer. Then, the first parameter may be compared to a first condition 574. The first condition may be, for example, a white blood cell concentration value of about 80%. If the first parameter meets or exceeds the first condition, then the workflow may continue. If the first parameter does not meet or exceed the first condition, the workflow may be stopped and/or an alert may be generated via the controller.

The method 505 may further comprise performing a second cell process and measuring one or more of a second parameter, a third parameter, and a fourth parameter 576. The second cell process may comprise selection. One or more of the second, third, and fourth parameters may be measured at a second time interval. For example, the second parameter may be a cell concentration value, such as a CD4+ and/or CD8+ concentration value. The CD4+ and/or CD8+ concentration value may be measured by a flow cytometer. The third parameter may be a cell recovery value, such as a CD4+ and/or CD8+ recovery value. The fourth parameter may be a target cell value. Then, one or more of the second, third, and fourth parameters may be compared to a second, third, and fourth condition, respectively 578. The second condition may be a CD4+ and/or CD8+ concentration value of about 90%. The third condition may be a CD4+ and/or CD8+ recovery value of about 30%. The fourth condition may be a target cell value of about 800E6 cells.

If the second parameter is less than the second condition and/or the third parameter is less than the third condition, then an alert may be generated via the controller. If both the second and third conditions are met or exceeded and the fourth parameter meets or exceeds the fourth condition, then a quantity of cells may be removed from the cell solution. The quantity of removed cells may correspond to the difference between the measured fourth parameter and the fourth condition, such that after removal the cell solution may contain a quantity of target cells below the threshold represented by the fourth condition. If none of the second, third, and fourth conditions are met or exceeded, then the workflow may continue.

The method 505 may further comprise performing a third cell process and measuring one or more of a fifth parameter and a sixth parameter 580. The third cell process may comprise activation. One or more of the fifth and sixth parameters may be measured at a third time interval. For example, the fifth parameter may be a cell diameter value. The sixth parameter may be a cell viability value. Then, one or more of the fifth and sixth parameters may be compared to a fifth and sixth condition, respectively 582. The fifth condition may be a cell diameter value of about 10 micrometers (μm). The sixth condition may be a cell viability value of about 70%. If the fifth parameter is less than the fifth condition and/or the sixth parameter is less than the sixth condition, then an alert may be generated via the controller. If both of the second and third conditions are met or exceeded, then the workflow may continue.

The method 505 may further comprise performing a fourth cell process and measuring one or more of a seventh parameter and an eighth parameter 584. The fourth cell process may comprise perfusion. One or more of the seventh and eighth parameters may be measured at a fourth time interval. For example, the seventh parameter may be a glucose value. The eighth parameter may be a lactate value. Then, one or more of the seventh and eighth parameters may be compared to a seventh and eighth condition, respectively 586. The fifth condition may be a glucose value of about 2 g/L. The eighth condition may be a lactate value of about 2 g/L. If the seventh parameter is less than the seventh condition and/or the eighth parameter is greater than the eighth condition, then the cell solution may be perfused to a pre-determined perfusion value. For example, the pre-determined perfusion value may be about 50%, such that about 50% of the media may be removed and replaced while retaining substantially all of the cells within the cell solution. If the seventh parameter meets or exceeds the seventh condition and the eighth parameter is less than the eighth condition, then the workflow may continue.

The method 505 may further comprise performing a fifth cell process and measuring one or more of a ninth parameter, a tenth parameter, and an eleventh parameter 588. The fifth cell process may comprise expansion and/or perfusion. One or more of the ninth, tenth, and eleventh parameters may be measured at a fifth time interval. For example, the ninth parameter may be a total cell count value. The tenth parameter may be a glucose value and the eleventh parameter may be a lactate value, similar to the fourth cell process described previously. Then, one or more of the ninth, tenth, and eleventh parameters may be compared to a ninth, tenth, and eleventh condition, respectively 590. The ninth condition may be a total cell count value of a pre-determined value. The pre-determined value may correspond to a historical average based on one or more prior workflows. If the ninth parameter is less than the ninth condition, then another sample may be obtained and measured. If the ninth parameter meets or exceeds the ninth condition, then one or more of the tenth and eleventh parameters may be compared to the ninth and tenth conditions, respectively. The fifth cell process may be similar to the fourth cell process, such that tenth parameter may be a glucose value and the eleventh parameter may be a lactate value. Similarly, the tenth condition may be equivalent to the seventh condition and the eleventh condition may be equivalent to the eight condition. Accordingly, if the tenth parameter is less than the tenth condition and/or the eleventh parameter is greater than the eleventh condition, then the cell solution may be perfused to a pre-determined perfusion value. If the tenth parameter meets or exceeds the tenth condition and the eleventh parameter is less than the eleventh condition, then the workflow may continue.

The method 505 may further comprise performing a sixth cell process and measuring one or more of a twelfth parameter and a thirteenth parameter 592. The sixth cell process may comprise perfusion. The sixth cell process may be similar to the fourth cell process, such that twelfth parameter may be a glucose value and the thirteenth parameter may be a lactate value. Similarly, the twelfth condition may be equivalent to the seventh condition and the thirteenth may be equivalent to the eight condition. The method 505 may comprise comparing one or more of the twelfth parameter to the twelfth condition and the thirteenth parameter to the thirteenth condition 594. Accordingly, if the twelfth parameter is less than the twelfth condition and/or the thirteenth parameter is greater than the thirteenth condition, then the cell solution may be perfused to a pre-determined perfusion value. If the twelfth parameter meets or exceeds the twelfth condition and the thirteenth parameter is less than the thirteenth condition, then the workflow may continue.

The method 504 may further comprise performing a seventh cell process and measuring one or more of a fourteenth parameter and a fifteenth parameter 595. The seventh cell process may comprise expansion. One or more of the fourteenth and fifteenth parameters may be measured at a seventh time interval. For example, the fourteenth parameter may be a total cell count value. The fifteenth parameter may be a chimeric antigen receptor expression value. Then, one or more of the fourteenth and fifteenth parameters may be compared to a fourteenth and fifteenth condition, respectively 596. The fourteenth condition may be a total cell count value of a pre-determined value. The pre-determined value may correspond to a historical average based on one or more prior workflows, such as about 4E9 cells. The fifteenth condition may be a chimeric antigen receptor expression value of about 10%. If the fourteenth parameter is greater than the fourteenth condition and/or the fifteenth parameter is greater than the fifteenth condition, then the workflow may continue. If the fourteenth parameter is less than the fourteenth condition and the fifteenth parameter is less than the fifteenth condition, then the expansion process may continue.

The method 505 may further comprise performing an eighth cell process 597. The eighth cell process may comprise harvesting the cells from the cell solution. In some variations, the cell solution may be transferred to the sterile liquid transfer instrument, such that at least a portion of the cell solution may be removed from the cartridge and subsequently transferred to an SLTD. The SLTD may be moved by the robot to a reagent vault for storage or the feedthrough for removal from the workcell. In this way, the contents may be saved for subsequent use in cell therapies and/or used for future workflows to grow additional cells.

While described above as containing certain steps, it should be understood that the methods of cell processing may include any subset of cell processing steps in any suitable order.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for cell processing comprising:
automatically executing a pre-programmed workflow defining a set of cell processing steps, wherein the execution of at least one of the cell processing steps comprises measuring at least one parameter based upon a set of pre-programmed conditions and corresponding outcomes for that step;
determining whether a condition has been met based upon the at least one measured parameter; and
continuing, modifying, or stopping the workflow based upon the determination.

2. The method of claim 1, wherein the set of cell processing steps comprises one or more of enrichment, selection, activation, expansion, perfusion, sampling, and harvesting.

3. The method of claim 1, wherein two or more cell processing steps are executed simultaneously.

4. The method of claim 1, wherein the parameter comprises one or more of a total cell count value, a target cell count value, a cell concentration value, a cell recovery value, a cell diameter value, a cell viability value, a glucose value, a lactate value, a dissolved oxygen value, a pH value, a chimeric antigen receptor expression value, and a transgenic T cell receptor expression value.

5. The method of claim 1, wherein the parameter is measured at a predetermined time interval.

6. The method of claim 1, wherein the condition defines a threshold value for the parameter.

7. The method of claim 1, wherein a cell processing step is executed upon a determination that the measured parameter meets the condition.

8. The method of claim 1, wherein cells are transferred from a first module to a second module upon a determination that the measured parameter meets the condition.

9. The method of claim 1, wherein a notification to a user is generated upon a determination that the measured parameter does not meet the condition.

10. The method of claim 1, wherein the workflow is stopped upon a determination that the measured parameter does not meet the condition.

11. The method of claim 1, wherein at least one cell processing step is repeated upon a determination that the measured parameter does not meet the condition.

12.-18. (canceled)

19. A method for automated cell processing comprising:
receiving a workflow, the workflow defining one or more cell processing steps to be performed on cells within a cartridge within a workcell, wherein the cell processing steps are selected from the group consisting of washing, isolating, purifying, enriching, diluting, and growing cells;
automatically executing the workflow;
automatically measuring at least one parameter within the cartridge, wherein the at least one parameter is selected from the group consisting of cell count, cell diameter, cell concentration, cell viability, glucose, lactate, cell recovery, dissolved oxygen, and pH;
comparing the at least one measured parameter to a pre-defined condition; and
automatically continuing, modifying, or stopping the workflow based upon the comparison.

20. The method of claim 19, wherein the workflow is automatically executed by a robot.

21. The method of claim 19, wherein the workflow is pre-programmed into a controller.

22. The method of claim 19, wherein the pre-defined condition is pre-programmed into a controller.

23. The method of claim 19, wherein the pre-defined condition defines a threshold value for the measured parameter.

24. The method of claim 19, wherein continuing the workflow comprises ending a first cell processing step and executing a second cell processing step.

25. The method of claim 19, wherein an alert is generated upon the comparison indicating that the measured parameter does not meet the pre-defined condition.

26. The method of claim 19, wherein the workflow is stopped upon the comparison indicating that the measured parameter does not meet the pre-defined condition.

27. The method of claim 19, wherein at least one cell processing step is repeated upon the comparison indicating that the measured parameter does not meet the pre-defined condition.

28. The method of claim 19, wherein the one or more cell processing steps are performed at one or more pre-defined time intervals.

29. The method of claim 19, wherein measuring at least one parameter is performed by an analytical instrument.

30. The method of claim 19, wherein a sample of the cells within the cartridge is transferred to an analytical instrument.

31. The method of claim 19, wherein the diluting the cells comprises demidepletion.

32.-39. (canceled)
